(19) United States
(12) Patent Application Publication
(10) Pub. No.: US 2022/0248780 A1
(43) Pub. Date: Aug. 11, 2022

(54) Facemask

(71) Applicant: Dan Benzakin, Lewes, DE (US)

(72) Inventor: Dan Benzakin, Lewes, DE (US)

(21) Appl. No.: 17/479,451

(22) Filed: Sep. 20, 2021

Related U.S. Application Data
(60) Provisional application No. 63/080,935, filed on Sep. 21, 2020.
(60) Provisional application No. 63/183,324, filed on May 3, 2021.

(51) Int. Cl. A41D 13/11; A62B 23/02; A62B 18/08

(52) U.S. Cl. CPC A41D 13/1184; A62B 18/086; A62B 23/02

(57) ABSTRACT

A facemask adapted to be positioned on a user's face may include multiple components: a lower component or nose/mouth component; and an upper component or eye shield component. The eye shield component may be configured to at least partially cover the eyes of the user, while the nose/mouth component may be configured to cover the nose and mouth of the user. The eye shield component may be removably coupled to the nose/mouth component. The nose/mouth component may be configured to filter aerosols and droplets from the air/gas inhaled and exhaled by the user. The nose/mouth component may further facilitate the consumption of food and/or beverages without requiring the facemask to be removed or pulled down/away from the user's face.

### CROSS-REFERENCE TO RELATED APPLICATIONS

This application claims priority under 35 U.S.C. 119(e) to U.S. Provisional Patent Application Ser. No. 63/080,935, entitled “Facemask”, filed Sep. 21, 2020, and U.S. Provisional Patent Application Ser. No. 63/183,324, entitled “Facemask”, filed May 3, 2021. The disclosures of the above applications are incorporated herein by reference in their entirety for all purposes.

### FIELD OF THE INVENTION

The present invention relates to an accessory article and, in particular, an accessory article that is a facemask that may be worn by a user, where the facemask includes a detachable eye shield.

### BACKGROUND OF THE INVENTION

Conventional facemasks that are often worn by users are made of fabric or other similar materials. Oftentimes, the materials that these conventional facemasks are made from do not provide adequate filtering of aerosols and droplets from inhaled and exhaled air/gas. In addition, given their construction and structure, the conventional facemasks often fit loosely on a user's face, allowing unfiltered exhaled air to escape around the edges of the mask. Furthermore, in order to consume beverages and/or food with these conventional facemasks, the conventional facemasks must be removed, pulled away from, or pulled down from the user's face, which fully exposes the users nose and mouth to the surrounding environment. These conventional facemasks also fail to provide any sort of protection to a user's eyes.

It would be desirable to provide a facemask configured to address the above described deficiencies.

### SUMMARY OF THE INVENTION

The present invention is directed toward a facemask adapted to be positioned on a user's face, where the facemask may include multiple components. The facemask may include both a lower component (i.e., nose/mouth component), and an upper component (i.e., eye shield component). The eye shield component may be configured to at least partially cover the eyes of the user, while the nose/mouth component may be configured to cover the nose and mouth of the user. The eye shield component may be removably coupled to the nose/mouth component. Furthermore, the nose/mouth component may fit over the nose and mouth of the user such that at least a partial seal is formed around the nose and mouth of the user by the nose/mouth component. The nose/mouth component may be further equipped with a filter and filter enclosure that is configured to filter aerosols and droplets from the air/gas inhaled and exhaled by the user. The filter enclosure may be repositionable with respect to the rest of the nose/mouth component to facilitate, along with a straw access and cover, the consumption of food and/or beverages without requiring the facemask to be removed or pulled down/away from the user's face.

In one embodiment, a facemask configured to be worn by a user includes a first portion and a second portion. The first portion may be configured to cover a nose and a mouth of the user when the facemask is worn by the user. The second portion may be removably coupled to the first portion such that the second portion may be positioned proximate to the eyes of the user when the second portion is coupled to the first portion and the facemask is worn by the user.

In some instances, the second portion is a transparent shield that may include at least one coupling element descending from a bottom edge of the second portion. In addition, the first portion may include at least one coupling member that may be configured to receive the at least one coupling element of the second portion when the second portion is removably coupled to the first portion. Furthermore, the at least one coupling member of the first portion may be constructed from a material that both may promote a degree of deformability of the at least one coupling member and may be configured to impart friction forces on the at least one coupling element received by the at least one coupling member when the second portion is removably coupled to the first portion.

In some further instances, the at least one coupling element may be a first coupling element descending from the bottom edge of the second portion proximate to a first side edge of the second portion. The second portion may further include a second coupling element descending from the bottom edge of the second portion proximate to a second side edge of the second portion, where the second side edge is opposite the first side edge. According to some instances, the bottom edge of the second portion may also include a central notch disposed between the first coupling element and the second coupling element such that the central notch may be disposed equidistant from the first side edge and the second side edge. In some even further instances, the at least one coupling member may be a first coupling member disposed on the first portion more proximate to a first side of the first portion than a second side of the first portion. The first portion may further include a second coupling member disposed on the first portion more proximate to the second side of the first portion than the first side of the first portion. The first coupling member may be configured to receive the first coupling element while the second coupling member may be configured to receive the second coupling element when the second portion is removably coupled to the first portion. According to some further instances, the first portion may further include a securement slot that may be disposed between the first coupling member and the second coupling member. The central notch of the second portion may be disposed within the securement slot when the second portion is removably coupled to the first portion.

In another embodiment, a facemask configured to be worn by a user includes a lower component and an upper component. The lower component may include a mouth portion and a nose portion disposed above the mouth portion. The mouth portion may be configured to cover a mouth of the user when the facemask is worn by the user. The nose portion may be configured to cover a nose of the user when the facemask is worn by the user. The upper component may be removably coupled to the nose portion of the lower component. The upper component may be positioned proximate to the eyes of the user when the upper component is coupled to the lower component and the facemask is worn by the user.

In some instances, the upper component is a transparent shield that may include at least one coupling element descending from a bottom edge of the upper component. In addition, the lower component may include at least one coupling member that may be configured to receive the at least one coupling element of the upper component when the upper component is removably coupled to the lower component. Furthermore, the at least one coupling member of the lower component may be constructed from a material that both may promote a degree of deformability of the at least one coupling member and may be configured to impart friction forces on the at least one coupling element received by the at least one coupling member when the upper component is removably coupled to the lower component.

In some further instances, the lower component may further include a central opening and a filter enclosure. The central opening may be disposed in the mouth portion of the lower component such that the central opening is disposed proximate to the mouth of the user when the facemask is worn by the user. The filter enclosure may be rotatably coupled to the mouth portion of the lower component proximate to the central opening. The filter enclosure may be rotatable between a closed position, where the filter enclosure may be disposed within the central opening, and an open position, where the filter enclosure may be rotated away from the central opening.

In some even further instances, the lower component may include a straw access opening and a cover. The straw access opening may be disposed in the mouth portion of the lower component, and the straw access opening may be configured to receive a straw. The cover may be coupled to the straw access opening, and may be reconfigurable between an uncovered position, where the user may insert the straw into the straw access opening, and a covered position, where the cover is disposed over the straw access opening.

In yet another embodiment, a facemask configured to be worn by a user includes a nose and mouth component and an eye shield component. The nose and mouth component may be configured to cover a nose and a mouth of the user when the facemask is worn by the user. The eye shield component may be removably coupled to the nose and mouth component. The eye shield component may be positioned proximate to the eyes of the user when the eye shield component is coupled to the nose and mouth component and the facemask is worn by the user.

In some instances, the eye shield component is transparent and may include at least one coupling element descending from a bottom edge of the eye shield component. In addition, the nose and mouth component may include at least one coupling member that may be configured to receive the at least one coupling element of the eye shield component when the eye shield component is removably coupled to the nose and mouth component. Furthermore, the at least one coupling member of the nose and mouth component may be constructed from a material that both may promote a degree of deformability of the at least one coupling member and may be configured to impart friction forces on the at least one coupling element received by the at least one coupling member when the eye shield component is removably coupled to the nose and mouth component.

In some further instances, the nose and mouth component may be constructed from an impermeable and waterproof material. According to other instances, the nose and mouth component may be constructed from a fabric and a flexible membrane disposed on an exterior surface of the fabric. According to even further instances, the nose and mouth component may be constructed from a mesh of synthetic polymer fibers.

The above and still further features and advantages of the present invention will become apparent upon consideration of the following detailed description of specific embodiments thereof.

### BRIEF DESCRIPTION OF THE DRAWINGS

FIG. 1 illustrates a front elevational view of an embodiment of a facemask according to the present invention.

FIG. 2 illustrates a side elevational view of the embodiment of the facemask illustrated in FIG. 1.

FIG. 3 illustrates a front view of an nose/mouth component of the embodiment of the facemask illustrated in FIG. 1, the nose/mouth component being equipped with a securement or attachment component for securing the facemask to the head of a user.

FIG. 4 illustrates a side elevational view of the nose bridge portion of the nose/mouth component of the embodiment of the facemask illustrated in FIG. 1.

FIG. 5 illustrates a cross-sectional view of a coupling member of the nose/mouth component of the embodiment of the facemask illustrated in FIG. 1 that is taken along line 5-5 in FIG. 1, the coupling member serving to couple the eye shield to the nose/mouth component of the facemask.

FIG. 6A illustrates a side elevational view of the filter enclosure of the nose/mouth component of the embodiment of the facemask illustrated in FIG. 1.

FIG. 6B illustrates a front elevational view of the embodiment of the facemask illustrated in FIG. 1 where the filter enclosure is repositioned to the open position.

FIG. 6C illustrates a front view of a filter of the embodiment of the facemask illustrated in FIG. 1.

FIG. 6D illustrates an exploded side view of the embodiment of the filter illustrated in FIG. 6C.

FIG. 7 illustrates a front elevational view of the eye shield component of the embodiment of the facemask illustrated in FIG. 1.

FIG. 8 illustrates an isolated view of a coupling element of the embodiment of the eye shield component illustrated in FIG. 7, the coupling element facilitating the removable coupling of the eye shield component from the nose/mouth component.

FIG. 9 illustrates a side view of the embodiment of the facemask illustrated in FIG. 1, and illustrating the coupling of the eye shield component to the nose/mouth component of the facemask illustrated in FIG. 1.

FIGS. 10A and 10B illustrate top and side views, respectively, of the embodiment of the facemask illustrated in FIG. 1 being worn by a user.

FIGS. 11A and 11B illustrate front and side views, respectively, of a second embodiment of a facemask according to the present invention.

FIG. 11C illustrates a cross-sectional view of a coupling member of the nose/mouth component and a coupling element of the eye shield component of the embodiment of the facemask illustrated in FIGS. 11A and 11B that is taken along line 11-11 in FIG. 11A, the coupling member and coupling element serving to couple the eye shield component to the nose/mouth component of the facemask.

FIGS. 12A and 12B illustrate front and side views, respectively, of a third embodiment of a facemask according to the present invention.

FIG. 12C illustrates a cross-sectional view of a coupling member of the nose/mouth component and a coupling element of the eye shield component of the embodiment of the facemask illustrated in FIGS. 12A and 12B that is taken along line 12-12 in FIG. 12A, the coupling member and coupling element serving to couple the eye shield component to the nose/mouth component of the facemask.

FIG. 13A illustrates a perspective view of the straw access of the embodiment of the facemask illustrated in FIGS. 12A and 12B, where the cover of the straw access is in the open position.

FIG. 13B illustrates a cross-sectional view of a straw being inserted into the straw access illustrated in FIG. 13A that is taken along line 13-13 in FIG. 13A.

FIG. 13C illustrates an exterior front view of the straw access illustrated in FIG. 13A, where the cover of the straw access is in the open position and the hinged door is in the closed position.

FIG. 13D illustrates an exterior front view of the straw access illustrated in FIG. 13A, where the cover of the straw access and the hinged door are in the open positions.

FIG. 13E illustrates an interior rear view of the straw access illustrated in FIG. 13A, where the hinged door is in the closed position.

FIGS. 14A and 14B illustrate front and side views, respectively, of a fourth embodiment of a facemask according to the present invention.

FIG. 14C illustrates a cross-sectional view of a coupling member of the nose/mouth component and a coupling element of the eye shield component of the embodiment of the facemask illustrated in FIGS. 14A and 14B that is taken along line 14-14 in FIG. 14A, the coupling member and coupling element serving to couple the eye shield component to the nose/mouth component of the facemask.

FIG. 15A illustrates a view of a hospital room where the hospital staff present in the hospital room are utilizing the embodiment of the facemask illustrated in FIGS. 11A and 11B.

FIG. 15B illustrates a view of military troops utilizing the embodiment of the facemask illustrated in FIGS. 11A and 11B.

FIG. 15C illustrates a view of a classroom where the children present in the classroom are utilizing the embodiment of the facemask illustrated in FIGS. 12A and 12B.

Like reference numerals have been used to identify like elements throughout this disclosure.

### DETAILED DESCRIPTION OF THE INVENTION

The present invention relates to a facemask adapted to be positioned on a user's face that include multiple components. The embodiment of the facemask disclosed herein may have at least a lower component (i.e., nose/mouth component), and an upper component (i.e., eye shield component). The eye shield component may be configured to at least partially cover the eyes of the user, while the nose/mouth component may be configured to cover the nose and mouth of the user. The eye shield component may be removably coupled to the nose/mouth component such that the eye shield component, if desired, may be removed from the nose/mouth component. Because the nose/mouth component covers the nose and mouth of the user and is equipped with a repositionable filter enclosure, the nose/mouth component may be configured to filter aerosols and droplets from the air/gas inhaled and exhaled by the user. The nose/mouth component, via the repositionable filter enclosure and a straw access, may further facilitate the consumption of food and/or beverages without requiring the facemask to be removed or pulled down/away from the user's face.

In the following detailed description, reference is made to the accompanying figures which form a part hereof wherein like numerals designate like parts throughout, and in which is shown, by way of illustration, embodiments that may be practiced. It is to be understood that other embodiments may be utilized, and structural or logical changes may be made without departing from the scope of the present disclosure. Therefore, the following detailed description is not to be taken in a limiting sense, and the scope of embodiments is defined by the appended claims and their equivalents.

Aspects of the disclosure are disclosed in the description herein. Alternate embodiments of the present disclosure and their equivalents may be devised without parting from the spirit or scope of the present disclosure. It should be noted that any discussion herein regarding “one embodiment”, “an embodiment”, “an exemplary embodiment”, and the like indicate that the embodiment described may include a particular feature, structure, or characteristic, and that such particular feature, structure, or characteristic may not necessarily be included in every embodiment. In addition, references to the foregoing do not necessarily comprise a reference to the same embodiment. Finally, irrespective of whether it is explicitly described, one of ordinary skill in the art would readily appreciate that each of the particular features, structures, or characteristics of the given embodiments may be utilized in connection or combination with those of any other embodiment discussed herein.

Various operations may be described as multiple discrete actions or operations in turn, in a manner that is most helpful in understanding the claimed subject matter. However, the order of description should not be construed as to imply that these operations are necessarily order dependent. In particular, these operations may not be performed in the order of presentation. Operations described may be performed in a different order than the described embodiment. Various additional operations may be performed and/or described operations may be omitted in additional embodiments.

For the purposes of the present disclosure, the phrase “A and/or B” means (A), (B), or (A and B). For the purposes of the present disclosure, the phrase “A, B, and/or C” means (A), (B), (C), (A and B), (A and C), (B and C), or (A, B and C).

The terms “comprising,” “including,” “having,” and the like, as used with respect to embodiments of the present disclosure, are synonymous.

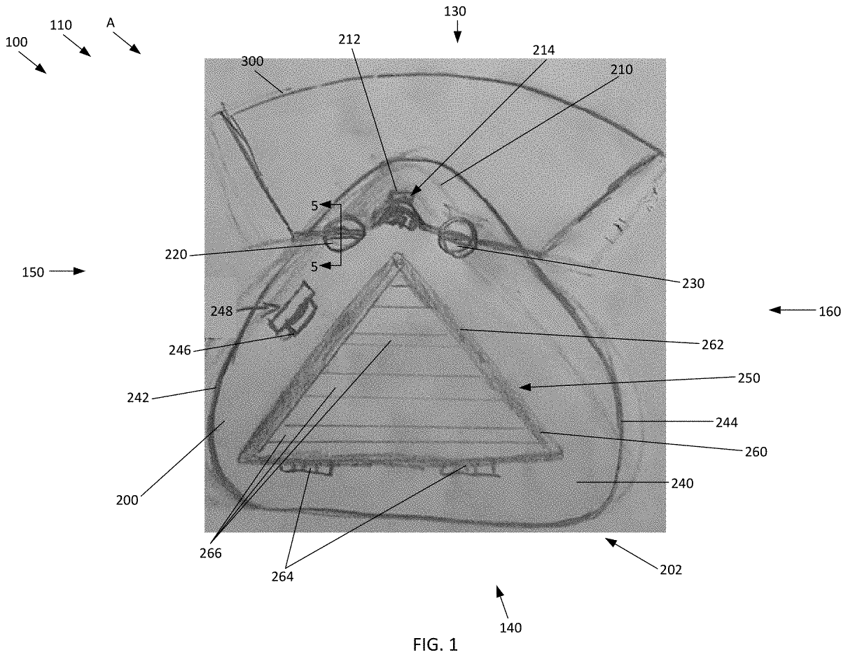

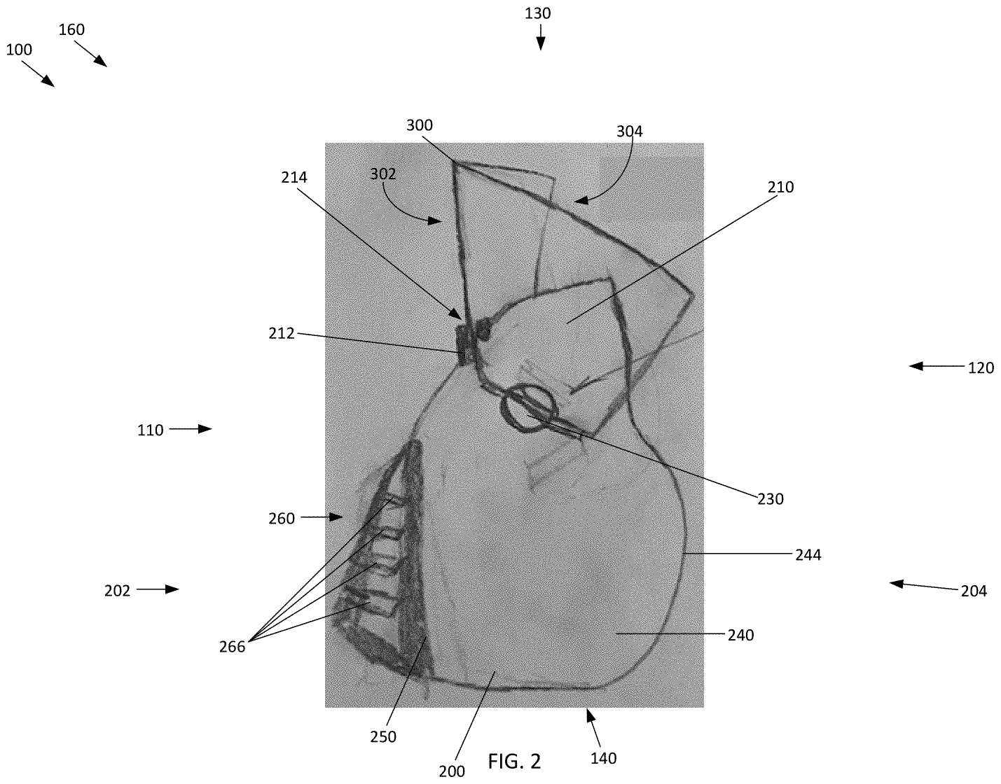

With general reference to FIGS. 1 and 2, a facemask 100 configured to be worn by a user is shown. As illustrated, the facemask 100 includes an front side or exterior side 110 and an opposite rear side or interior side 120. When the facemask 100 is worn by a user, the interior side 120 may primarily face toward the user, while the exterior side 110 may primarily face away from the user. The facemask 100 may further include a top side 130, an opposite bottom side 140, a first side 150, and an opposite second side 160. As further illustrated in FIGS. 1 and 2, the facemask 100 may include a first component 200, also referred to herein as a lower component or nose/mouth component, and a second component 300, also referred to herein as an upper component or eye shield component. As explained in further detail below, the nose/mouth component 200 is configured to cover the nose, mouth, and surrounding area of a user when the facemask 100 is worn by the user, while the eye shield component 300 is configured to be disposed proximate to and/or cover the eyes of the user.

As illustrated in FIGS. 1, 2, and 3, the nose/mouth component 200 includes an exterior side 202 and an opposite interior side 204. The exterior side 202 may primarily face away from the user, while the interior side 204 may primarily face toward the user when the facemask 100 is worn by the user. As best illustrated in FIG. 1, the nose/mouth component 200 may be substantially triangular in shape such that the nose/mouth component 200, when the facemask 100 is worn by a user, covers the nose and mouth area of a user. The nose/mouth component 200 includes a nose portion 210 and a mouth portion 240. The nose portion 210 may be located near the top side of the nose/mouth component 200 proximate to, and including, the top vertex/apex of the nose/mouth component 200. The mouth portion 240 may be located near the bottom side of the nose/mouth component 200 proximate to, and including, the bottom two vertices/apexes of the nose/mouth component 200 (and also proximate to the bottom side 140 of the facemask 100). In other embodiments, the nose/mouth component 200 may be formed in any other shape. Furthermore, the nose/mouth component 200 may be formed of any material that is waterproof and impermeable such as, but not limited to, rubber, synthetic rubber, silicone, polyvinyl chloride, polyurethane, etc.

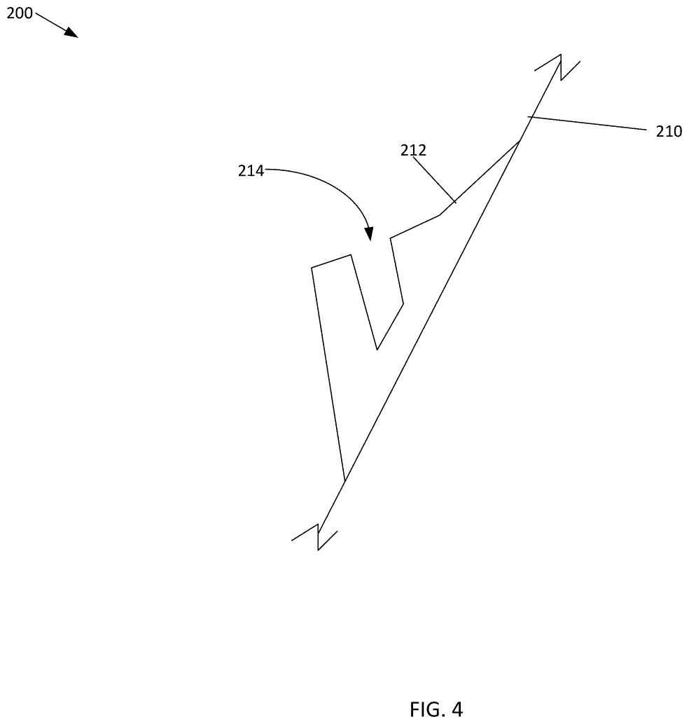

Continuing with FIGS. 1, 2, and 3, and with further reference to FIG. 4, the nose portion 210 of the nose/mouth component 200 may include a nose bridge 212 that includes a securement slot 214. The securement slot 214, as best illustrated in FIG. 4, may be a linear slot that extends through the nose bridge 212 of the nose portion 210 of the nose/mouth component 200 in a transverse direction to the facemask 100 (i.e., the securement slot 214 extends across the nose bridge 212 in a direction spanning from the first side 150 of the facemask 100 to the second side 160 of the facemask 100). As explained in further detail below, the securement slot 214 may be configured to receive a portion of the eye shield component 300 when the eye shield component 300 is coupled to the nose/mouth component 200. The nose bridge 212 may be constructed from rubber such that the nose bridge 212 has a degree of resiliency or deformability, while also being capable of forming a friction fit with the portion of the eye shield component 300 disposed within the securement slot 214 (i.e., the rubber substance of the nose bridge 212 may impart friction forces onto a portion of the eye shield component 300 to prevent the eye shield component 300 from shifting/moving with respect to the nose/mouth component 200). In other embodiments, the nose bridge 212 may be constructed from other materials that also provide a similar degree of resiliency or deformability to rubber, and are also capable of imparting friction forces onto a portion of the eye shield component 300.

With continued reference to FIGS. 1, 2, and 3, and with further reference to FIG. 5, the nose portion 210 may further include a first coupling member 220 and a second coupling member 230. The first coupling member 220 may be disposed on one side of the nose bridge 212, while the second coupling member 230 may be disposed on the opposing side of the nose bridge 212. Thus, the first coupling member 220 may be disposed more proximate to the first side 150 of the facemask 100 than the second coupling member 230, while the second coupling member 230 may be disposed more proximate to the second side 160 of the facemask 100 than the first coupling member 220. While FIG. 5 only illustrates a single coupling member, because coupling members 220, 230 are substantially similar to one another, the illustrated cross-section and associated description applies to both coupling members 220, 230. As illustrated in FIG. 5, the coupling members 220, 230 may each include an inverted T-shaped channel 222, 232. The channels 222, 232 may span or extend through the coupling members 220, 230, respectively, in a transverse direction to the facemask 100 (i.e., the channels 222, 232 extend through the coupling members 220, 230, respectively, in a direction spanning from the first side 150 of the facemask 100 to the second side 160 of the facemask 100). As explained in further detail below, the channels 222, 232 of the coupling members 220, 230 may be configured to receive portions of the eye shield component 300 when the eye shield component 300 is coupled to the nose/mouth component 200. Moreover, the coupling members 220, 230 may be constructed from rubber such that the coupling members 220, 230 have a degree of resiliency or deformability, while also being capable of forming a friction fit with the portions of the eye shield component 300 disposed within the channels 222, 232 of the coupling members 220, 230, respectively (i.e., the rubber substance of the coupling members 220, 230 may impart friction forces onto portions of the eye shield component 300 to prevent the eye shield component 300 from shifting/moving with respect to the nose/mouth component 200). In other embodiments, like the nose bridge 212, the coupling members 220, 230 may be constructed from other materials that also provide a similar degree of resiliency or deformability to rubber, and are also capable of imparting friction forces onto portions of the eye shield component 300.

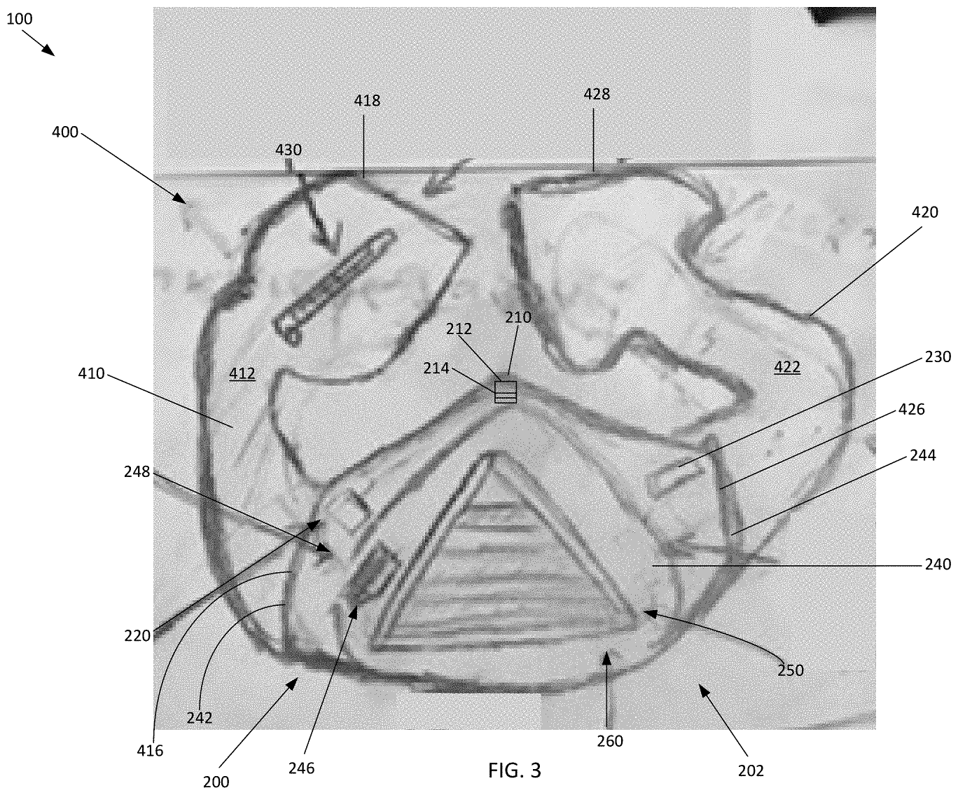

With continued reference to FIGS. 1, 2, and 3, and as previously explained, the nose/mouth component 200 further includes a mouth portion 240. As best illustrated in FIGS. 1 and 3, the mouth portion 240 include a first side 242 and an opposite second side 244. FIGS. 1 and 3 further illustrate that the mouth portion 240 includes an offset opening or straw access 246 that may be disposed on the mouth portion 240 more proximate to the first side 242 than the second side 244 of the mouth portion 240. The straw access 246 may span from the exterior side 202 of the nose/mouth component 200, through the nose/mouth component 200, to the interior side 204 of the nose/mouth component 200. Furthermore, the straw access 246 may be sized to receive a straw of a drink to enable a user of the facemask 100 to consume a beverage without having to remove the facemask 100 from their face or pull the facemask 100 away from their face. The mouth portion 240 may further include a cover 248 for the straw access 246. The cover 248 may be a hinged cover 248, may be secured to the mouth portion 240 or the straw access 246 via a tether, lanyard, or strap (not shown), or may simply be removable from the mouth portion 240. Furthermore, the cover 248 may be configured snap into/onto the straw access 246, or may be configured to form a friction fit within the straw access 246. The cover 248 may be constructed from rubber or other similar impermeable material.

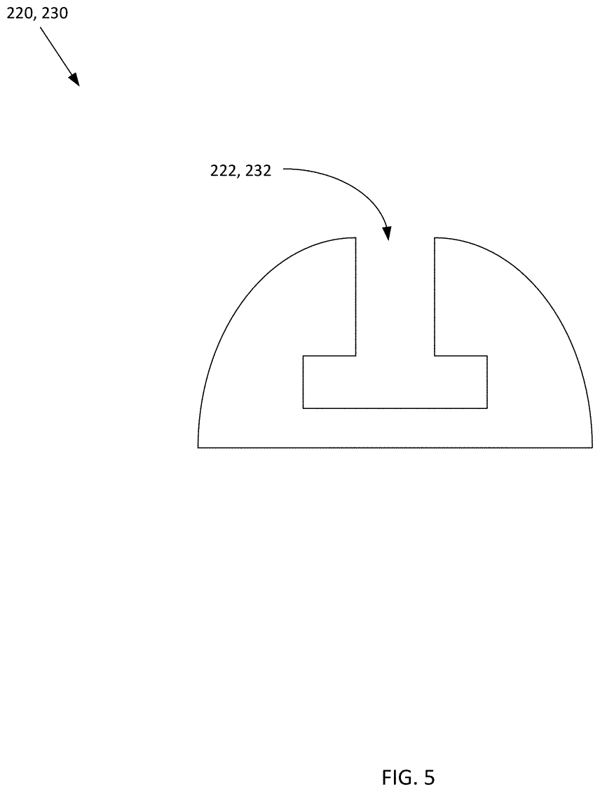

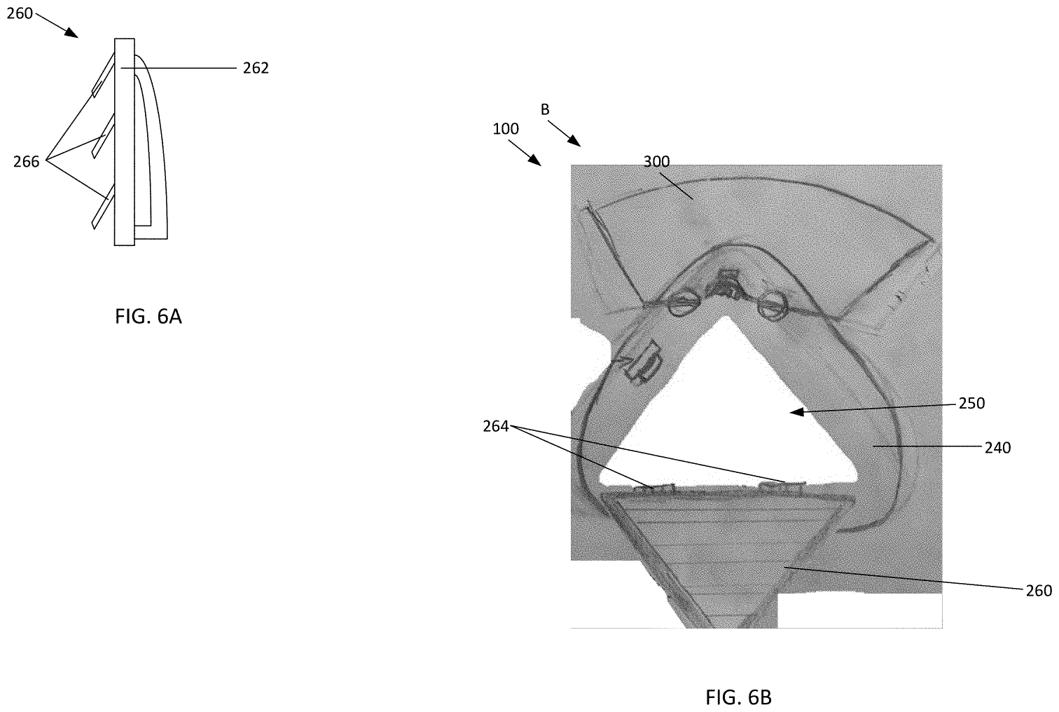

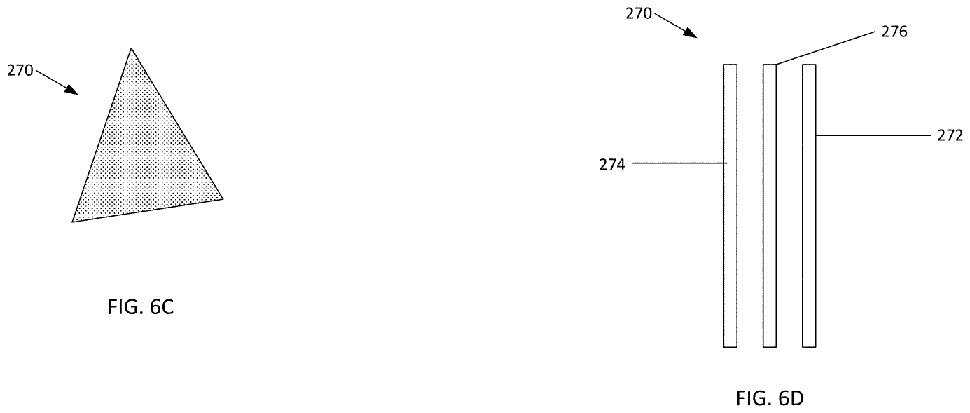

The mouth portion 240 may further include a central opening 250 and a filter enclosure 260 coupled to the mouth portion 240, and configured to fit within the central opening 250. In the embodiment illustrated in FIGS. 1, 2, and 3, the central opening 250 and the filter enclosure 260 are substantially triangular in shape. In other embodiments, the central opening 250 and the filter enclosure 260 may be of any other shape including, but not limited to, circular, square, pentagon, etc. The central opening 250 may span from the exterior side 202 of the nose/mouth component 200, through the mouth portion 240 of the nose/mouth component 200, and to the interior side 204 of the nose/mouth component 200. In the embodiment illustrated in FIGS. 1, 2, and 3, and further illustrated in FIG. 6A, the filter enclosure 260 may include a frame or structure 262 that is configured to house a filter 270, which is best illustrated in FIGS. 6C and 6D. The filter enclosure 260 may be rotatably coupled to the mouth portion 240 via a set of hinges 264 that are coupled to both the mouth portion 240 and the frame 262 of the filter enclosure 260. The hinges 264 enable the filter enclosure 260 to rotate about an axis extending through the hinges 264 such that the filter enclosure 260 is repositionable between a closed position A, as shown in FIGS. 1, 2, and 3, and an open position B, as shown in FIG. 6B. When in the closed position A, the filter enclosure 260 is disposed within the central opening 250 of the mouth portion 240. The filter enclosure 260 may snap into the central opening 250 of the mouth portion 240, or may be configured to frictionally fit within the mouth portion 240. As further illustrated in FIGS. 1, 2, 3, and 6A, the filter enclosure 260 may be further equipped with a series of baffles 266. In the illustrated embodiment, and as best illustrated in FIG. 6A, the baffles 266 may be angled downwardly with respect to a horizontal plane. In other embodiments, the baffles 266 may be adjustable such that their position and/or angle may be adjusted by a user of the facemask 100.

As previously explained, the filter 270, as illustrated in FIGS. 6B and 6C, may be configured to fit within the filter enclosure 260. As best illustrated in FIG. 6C, the filter 270 may be constructed from multiple layers, where, the embodiment illustrated, includes an inner layer 272, and outer layer 274, and an intermediate layer 276 disposed between the inner and outer layers 272, 274. The inner layer 272, outer layer 274, and intermediate layer 276 may be constructed from the same materials, or may all be constructed from differing materials. The filter 270 may be configured to enable a person to breath when wearing the facemask 100 and when the filter enclosure 260 is in the closed position, while simultaneously being configured to filter out aerosols and droplets from the air/gas both exhaled and inhaled by the user wearing the facemask 100. The baffles 266 of the filter enclosure 260 may further be configured to direct any user exhaled air/gas that passes through the filter 270 downward with respect to the facemask 100 and the user wearing the facemask 100. Directing air/gas that passes through the filter 270 in a downward direction may also aid in reducing the amount of fog that may build up on the eye shield component 300.

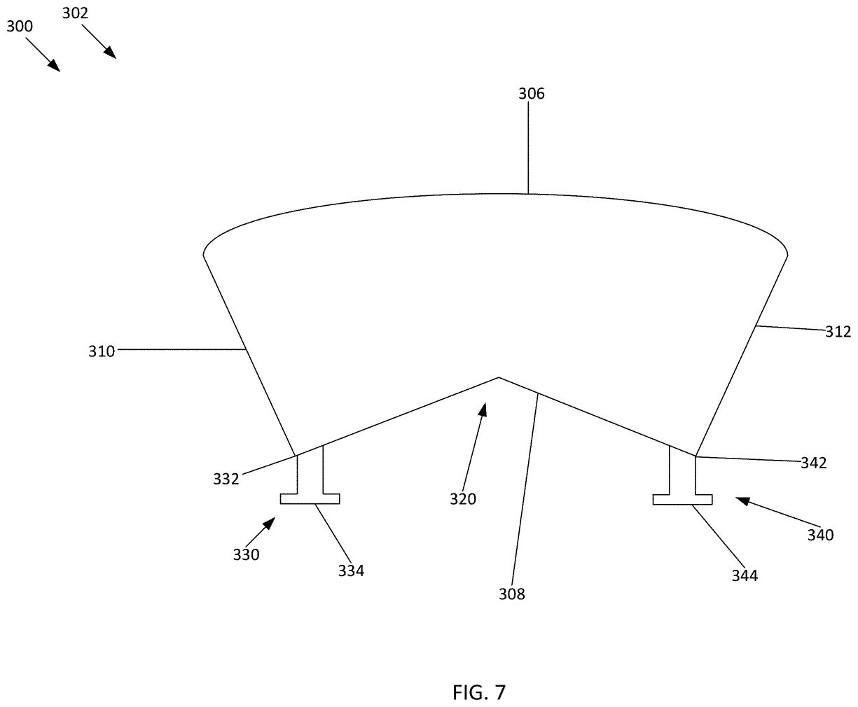

As previously explained, the facemask 100 includes an eye shield component 300 that is removably coupled to the nose/mouth component 200 of the facemask 100. As best illustrated in FIG. 7, the eye shield component 300 includes an exterior surface 302, which faces away from the user when the user is wearing the facemask 100, and an opposite interior surface 304, which faces toward the user when the user is wearing the facemask 100. The eye shield component 300 may further include a top edge 306, a bottom edge 308 opposite the top edge 306, a first side edge 310, and a second side edge 312 opposite the first side edge 310. The eye shield component 300 may have a transparency that enables a user to see objects and the surrounding environment through the eye shield component 300. The eye shield component 300 may be constructed from polycarbonate, plastic, or any other similar and suitable material.

As best illustrated in FIG. 7, the bottom edge 308 of the eye shield component 300 may contain a central notch or cutout portion 320 that is disposed along the bottom edge 308 equidistant from the first and second side edges 310, 312 of the eye shield component 300. The bottom edge 308 of the eye shield component 300 may further include a first coupling element 330 and a second coupling element 340. As best illustrated in FIG. 7, the first coupling element 330 is disposed more proximate to the first side edge 310 of the eye shield component 300 than the second side edge 312 of the eye shield component 300, while the second coupling element 340 is disposed more proximate to the second side edge 312 than the first side edge 310. As further illustrated in FIG. 7, the first and second coupling elements 330, 340 may descend from the bottom edge 308 of the eye shield component 300.

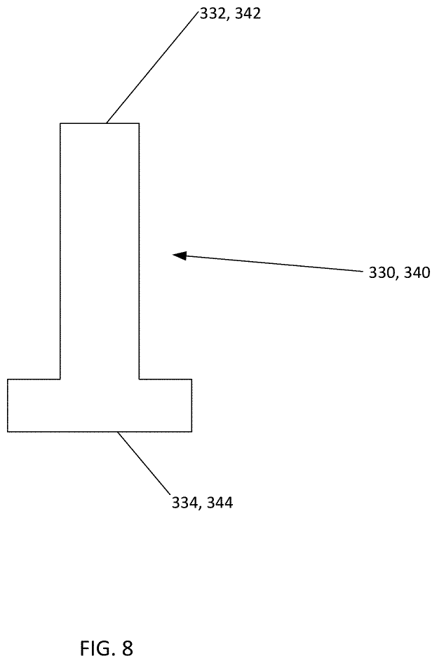

Turning to FIG. 8, illustrated is an isolated view of an example embodiment of a coupling element. While only one coupling element is illustrated in FIG. 8, because coupling elements 330, 340 are substantially similar to one another, the coupling element illustrated in FIG. 8 and associated description applies to both coupling elements 330, 340. As illustrated in FIGS. 7 and 8, the coupling elements 330, 340 may be elongated, where each coupling element 330, 340 may have having a proximal end 332, 342, respectively, and a distal end 334, 344, respectively. As illustrated in FIG. 7, the proximal ends 332, 342 of the coupling elements 330, 340, respectively, may be coupled to the bottom edge 308 of the eye shield component 300. Moreover, as best illustrated in FIG. 8, the distal ends 334, 344 of the coupling elements 330, 340, respectively, may be in the shape of an inverted T, which is similar in shape to that of the channels 222, 232 of the coupling members 220, 230 of the nose/mouth component 200. Thus, the distal ends 334, 344 of the coupling elements 330, 340 may be received by the channels 222, 232 of the coupling members 220, 230, respectively, to removably couple the eye shield component 300 to the nose/mouth component 200.

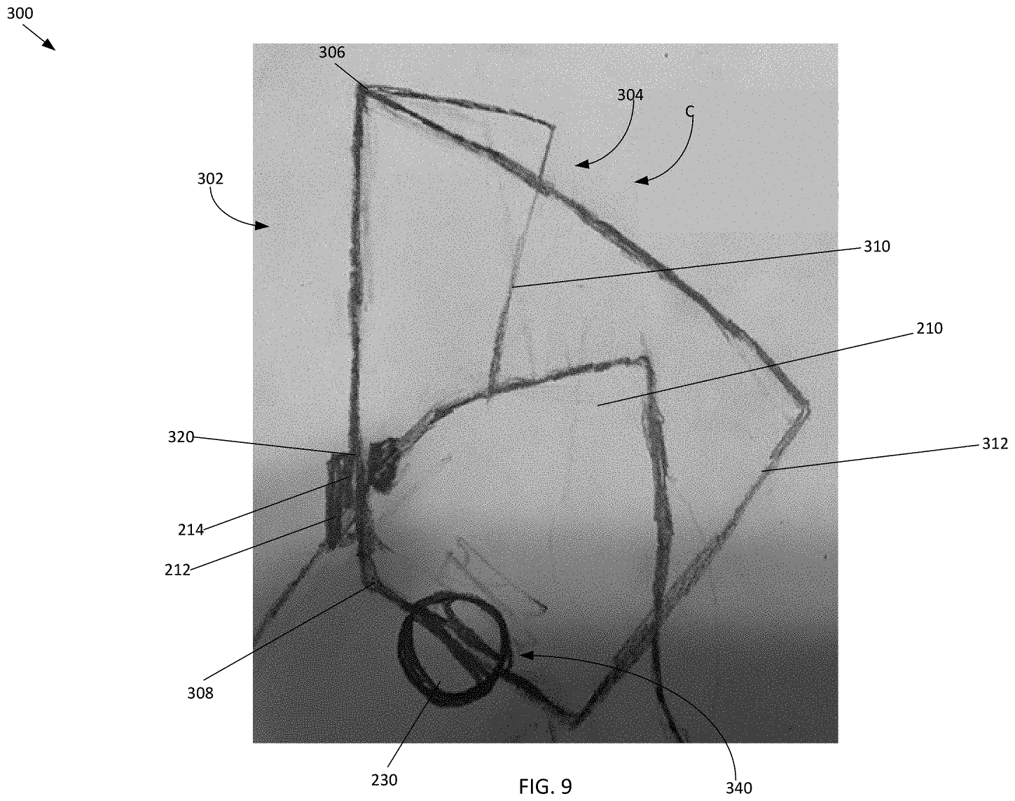

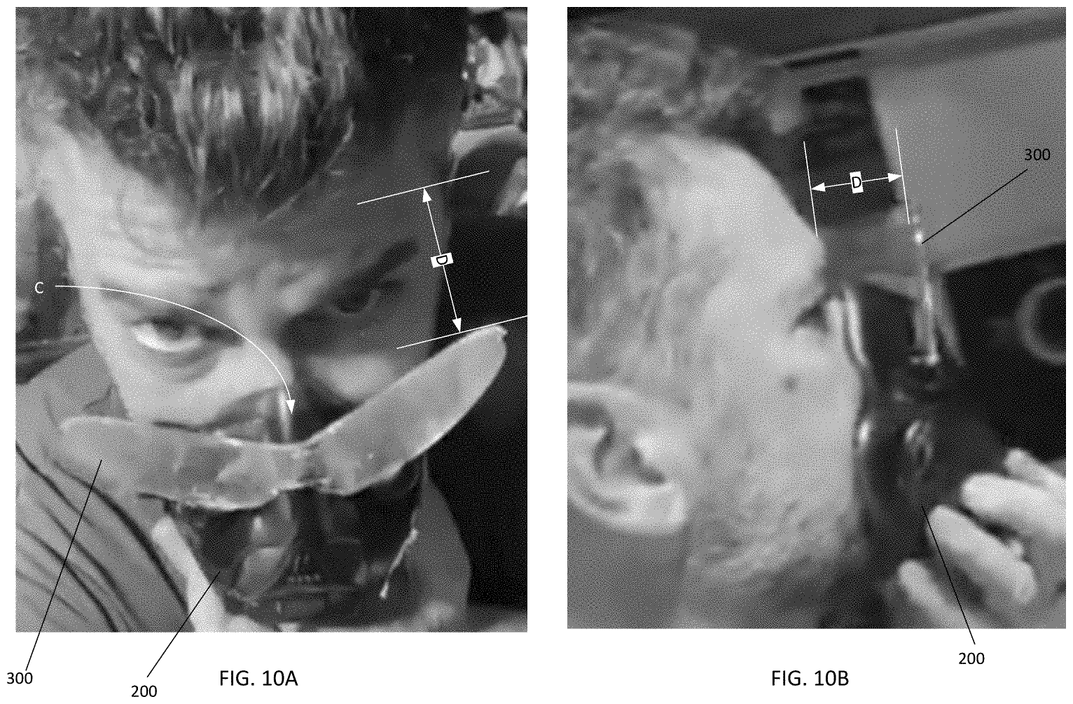

Turning to FIG. 9, illustrated is the eye shield component 300 coupled to the nose/mouth component 200 of the facemask 100. As previously explained, when the eye shield component 300 is coupled to the nose/mouth component 200, the distal ends 334, 344 of the coupling elements 330, 340 are received, respectively, by the channels 222, 232 of the coupling members 220, 230. Moreover, as illustrated in FIG. 9, the notch 320 of the bottom edge 308 of the eye shield component 300 is received within the slot 214 of the nose bridge 212. With the eye shield component 300 coupled to three attachment points (the two coupling members 220, 230 and the slot 214 of the nose bridge 212), the eye shield component 300 may remain secured to the nose/mouth component 200 without moving or shifting with respect to the nose/mouth component 200 during use of the facemask 100. As illustrated in FIG. 9, and further illustrated in FIG. 10A, the eye shield component 300 may have a curvature C such that the eye shield component 300 curves around the face of the user wearing the facemask 100. In other words, the eye shield component 300 may have a curvature C that is similar to the curvature of the face of a user of the facemask 100. Moreover, as further illustrated in FIGS. 10A and 10B, when the facemask 100 is worn by a user, the nose/mouth component 200 covers the nose and mouth of the user, while the eye shield component 300 is disposed in front of the eyes of the user such that the user looks through the eye shield component 300. Furthermore, the eye shield component 300 may be coupled to the nose/mouth component 200 such that, when the facemask 100 is worn by the user, the eye shield component 300 is spaced a distance D from the eyes of the user. The distance D may be a distance that is sufficiently far from the eyes and face of the user to prevent or reduce fogging of the eye shield component 300, while also being located close enough to the eyes of the user to provide protection to the eyes of the user. In addition, the distance D between the face of the user of the facemask 100 and the eye shield component 300 may be of a distance that enables a user to wear glasses while simultaneously wearing the facemask 100 without the eye shield component 300 interfering or contacting the glasses of the user.

Returning to FIG. 3, the facemask 100 may be further equipped with an attachment component 400. In the embodiment illustrated in FIG. 3, the attachment component 400 may include a first portion 410 and a second portion 420. The first portion 410 of the attachment component 400 may include an exterior surface 412 that faces away from the user, and an opposite interior surface 414 that faces toward the user when wearing the facemask 100. The first portion 410 of the attachment component 400 may further include a proximal end 416, which may be coupled to the first side 242 of the mouth portion 240 of the nose/mouth component 200, and an opposite distal end 418. Similar to the first portion 410, the second portion 420 of the attachment component 400 may include an exterior surface 422 that faces away from the user, and an opposite interior surface 424 that faces toward the user when wearing the facemask 100. The second portion 420 of the attachment component 400 may also include a proximal end 426, which may be coupled to the second side 244 of the mouth portion 240 of the nose/mouth component 200, and an opposite distal end 428. The first and second portions 410, 420 may be constructed from the same material as the nose/mouth component 200, or may be constructed from other materials, such as, but not limited to, fabric, synthetic fabrics, elastomeric materials, etc. When the facemask 100 is worn by a user, the first portion 410 may wrap around one side of the head of the user, while the second portion 420 may wrap around the opposite side of the head of the user such that the distal end 418 of the first portion 410 removably couples to the distal end 428 of the second portion 420. The distal end 418 of the first portion 410 and the distal end 428 of the second portion 420 may be equipped with fasteners to removably couple the two distal ends 418, 428 together. The fasteners may include, but are not limited to, hook and loop fasteners, snap button fasteners, button and slot fasteners, zipper fasteners, toggle and loop fasteners, etc. As further illustrated in FIG. 3, the first portion 410 of the attachment component 400 may further include a storage compartment 430 disposed proximate to the distal end 418 of the first portion 410. This storage compartment 430 may be configured to store items such as, but not limited to, additional filters, keys, credit cards, money, etc. In even further embodiments, the attachment component 400 may be elastic straps that the user may slip over their head to secure the facemask 100 to their head, or may be separate straps that the user may tie together behind their head to secure the facemask 100 to their head. In yet even further embodiments, the attachment component 400 may be a pair of looped straps on each side 150, 160 of the facemask 100 that are configured to wrap around the ears of the user to secure the facemask 100 to the head of the user.

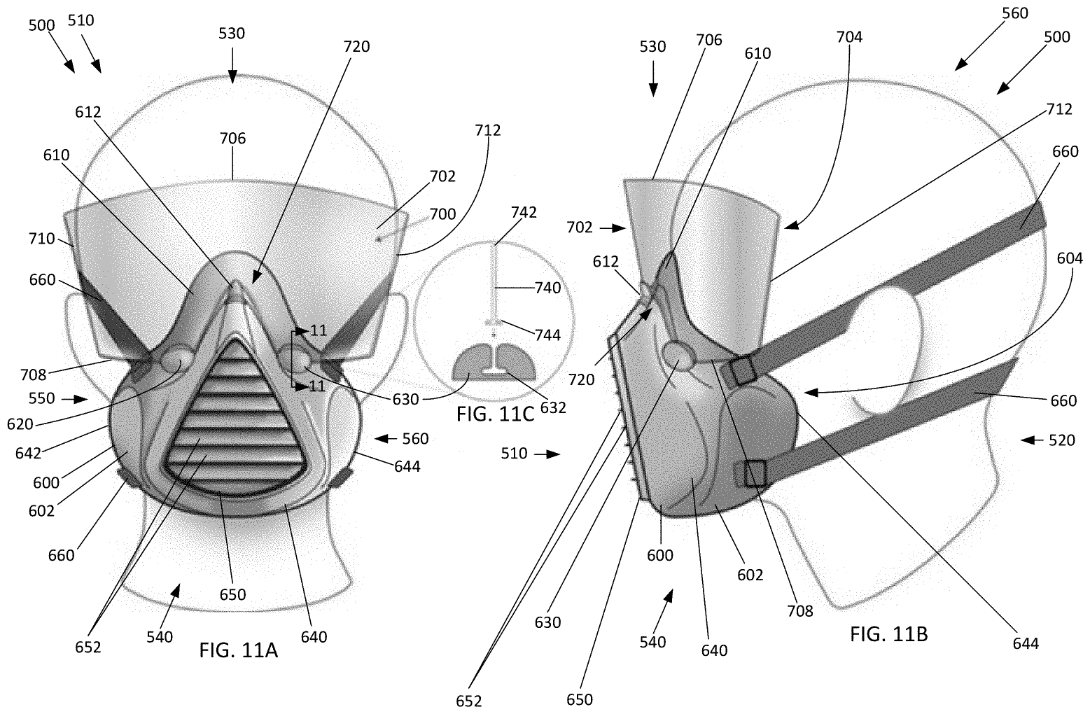

With general reference to FIGS. 11A and 11B, illustrated is a second embodiment of a facemask 500 configured to be worn by a user. The second embodiment of the facemask 500 may be similar to the first embodiment of the facemask 100 illustrated generally in FIGS. 1, 2, and 3. As illustrated in FIGS. 11A and 11B, the facemask 500 includes an front side or exterior side 510 and an opposite rear side or interior side 520. Like the first embodiment of the facemask 100, when the second embodiment of the facemask 500 is worn by a user, the interior side 520 may primarily face toward the user, while the exterior side 510 may primarily face away from the user. The second embodiment of the facemask 500 may further include a top side 530, an opposite bottom side 540, a first side 550, and an opposite second side 560. As further illustrated in FIGS. 11A and 11B, the facemask 500 may include a first component 600, also referred to herein as a lower component or nose/mouth component, and a second component 700, also referred to herein as an upper component or eye shield component.

The nose/mouth component 600 may be configured to cover the nose, mouth, and surrounding facial area of a user when the facemask 500 is worn by the user. As illustrated in FIGS. 11A and 11B, the nose/mouth component 600 includes an exterior side 602 and an opposite interior side 604. The exterior side 602 may primarily face away from the user, while the interior side 604 may primarily face toward the user when the facemask 500 is worn by the user. Like the first embodiment of the facemask 100, the nose/mouth component 600 of the second embodiment of the facemask 500 includes a nose portion 610 and a mouth portion 640. The nose portion 610 may be located near the top side of the nose/mouth component 600 proximate to, and including, the top vertex/apex of the nose/mouth component 600. The mouth portion 640 may be located near the bottom side of the nose/mouth component 600 proximate to, and including, the bottom two vertices/apexes of the nose/mouth component 600 (and also proximate to the bottom side 540 of the second embodiment of the facemask 500). While the nose/mouth component 600 illustrated in FIGS. 11A and 11B may be formed a rubber, synthetic rubber, or rubber-like material, the nose/mouth component 600 may be constructed/formed of any material that is waterproof and substantially impermeable including, but not limited to, silicone, polyvinyl chloride, polyurethane, etc.

Like the first embodiment of the facemask 100, as illustrated in FIGS. 11A and 11B, the nose portion 610 of the nose/mouth component 600 of the second embodiment of the facemask 500 may include a nose bridge 612 that includes a securement slot 614. The securement slot 614 may be similar to the securement slot 214 of the nose bridge 212 of the first embodiment of the facemask 100 that is illustrated in FIG. 4 in that the securement slot 614 may be a linear slot that extends through the nose bridge 612 of the nose portion 610 of the nose/mouth component 600 in a transverse direction to the facemask 500. In other words, the securement slot 614 may extend across the nose bridge 612 in a direction spanning generally from the first side 550 of the facemask 500 to the second side 560 of the facemask 500. The securement slot 614 may be configured to receive a portion of the eye shield component 700, as explained in further detail below, when the eye shield component 700 is coupled to the nose/mouth component 600. While the nose/mouth component 600 may be constructed from a rubber, synthetic rubber, or other rubber-like material, the nose bridge 612 may also be constructed from rubber or a rubber-like material such that the nose bridge 612 has a degree of resiliency or deformability. This degree of resiliency or deformability may enable the nose bridge 612 to form a friction fit with the portion of the eye shield component 700 disposed within the securement slot 614. In other words, the material of the nose bridge 612 may impart friction forces onto a portion of the eye shield component 700 to prevent the eye shield component 700 from shifting/moving with respect to the nose/mouth component 600.

With continued reference to FIGS. 11A and 11B, and with further reference to FIG. 11C, the nose portion 610 of the second embodiment of the facemask 500 may further include a first coupling member 620 and a second coupling member 630 like that of the first embodiment of the facemask 100. The first coupling member 620 may be disposed on one side of the nose bridge 612, while the second coupling member 630 may be disposed on the opposing side of the nose bridge 612. Thus, the first coupling member 620 may be disposed more proximate to the first side 550 of the facemask 500 than the second coupling member 630, while the second coupling member 630 may be disposed more proximate to the second side 560 of the facemask 500 than the first coupling member 620. While FIG. 11C only illustrates a single coupling member, because coupling members 620, 630 are substantially similar to one another, the illustrated cross-section and associated description applies to both coupling members 620, 630. As illustrated in FIG. 11C, the coupling members 620, 630 may each include an inverted T-shaped channel 622, 632. The channels 622, 632 may span or extend through the coupling members 620, 630, respectively, in a transverse direction to the facemask 500 (i.e., the channels 622, 632 extend through the coupling members 620, 630, respectively, in a direction spanning generally from the first side 550 of the facemask 500 to the second side 560 of the facemask 500). Like that explained previously for the channels 222, 232 of the coupling members 220, 230 of the first embodiment of the facemask 100, the channels 622, 632 of the coupling members 620, 630 may be configured to receive portions of the eye shield component 700 when the eye shield component 700 is coupled to the nose/mouth component 600. Like the nose bridge 612, the coupling members 620, 630 may be constructed from rubber or a similar material such that the coupling members 620, 630 have a degree of resiliency or deformability, while also being capable of forming a friction fit with the portions of the eye shield component 700 disposed within the channels 622, 632 of the coupling members 620, 630, respectively. In other words, the material of the coupling members 620, 630 may impart friction forces onto portions of the eye shield component 700 to prevent the eye shield component 700 from shifting/moving with respect to the nose/mouth component 600.

With continued reference to FIGS. 11A and 11B, the nose/mouth component 600 further includes a mouth portion 640, which includes a first side 642 and an opposite second side 644. The mouth portion 640 may further include a central opening/filter enclosure 650 disposed within the mouth portion 640. The central opening/filter enclosure 650 may be similar to central opening 250 and the filter enclosure 260 of the first embodiment of the facemask 100 in that the central opening/filter enclosure 650 of the second embodiment of the facemask 500 may be configured receive and house a filter (not show but similar to the filter 270 of the first embodiment of the facemask 100) that is configured to filter out aerosols and droplets from air passing through the filter. Moreover, the central opening/filter enclosure 650 may have components that are rotatably coupled to the mouth portion 640 such that those components may be rotated with respect to the mouth portion 640 to expose the mouth and/or interior 604 of the nose/mouth component 600. The central opening/filter enclosure 650 may be further equipped with one or more baffles 652 that may be angled downwardly with respect to a horizontal plane, or may be adjustable such that their position and/or angle may be adjusted by a user of the facemask 500. The baffles 652 may further be configured to direct any user exhaled air/gas that passes through the central opening/filter enclosure 650 downward with respect to the facemask 500 and the user wearing the facemask 500, which may aid in reducing the amount of fog that builds up on the eye shield component 700.

With further reference to FIGS. 11A and 11B, the second embodiment of the facemask 500 may include one or more straps 660. The straps 660 may be coupled to the nose/mouth component 600 on the first and second sides 642, 644 of the mouth portion 640 of the facemask 500. Thus, as illustrated in FIGS. 11A and 11B, when the facemask 500 is worn by a user, the straps 660 may be configured to wrap, at least partially, around the head of the user to removably secure the facemask 500 to the user's face. The straps 660 may contain a degree of resiliency such that they are capable of stretching, and/or their length may be adjustable so that the fit of the facemask 500 on the user's face/head may be made comfortable.

Continuing with FIGS. 11A and 11B, the second embodiment of the facemask 500, as previously explained, may include an eye shield component 700 that is removably coupled to the nose/mouth component 600 of the facemask 500. The eye shield component 700 may include an exterior surface 702, which faces away from the user when the user is wearing the facemask 500, and an opposite interior surface 704, which faces toward the user when the user is wearing the facemask 500. Like the eye shield component 300 of the first embodiment of the facemask 100, the eye shield component 700 of the second embodiment of the facemask 500 may further include a top edge 706, a bottom edge 708 opposite the top edge 706, a first side edge 710, and a second side edge 712 opposite the first side edge 710. The eye shield component 700 may have a transparency that enables a user to see objects and the surrounding environment through the eye shield component 700. The eye shield component 700 may be constructed from polycarbonate, plastic, or any other similar and suitable material.

Like the eye shield component 300 of the first embodiment of the facemask 100, the bottom edge 708 of the eye shield component 700 of the second embodiment of the facemask 500 may contain a central notch or cutout portion 720 that is disposed along the bottom edge 708 equidistant from the first and second side edges 710, 712 of the eye shield component 700. The bottom edge 708 of the eye shield component 700 may further include a first coupling element 730 and a second coupling element 740 that descend from the bottom edge 708 of the eye shield component 700. As best illustrated in FIG. 11C, which is a cross-sectional view of the coupling member 630 and the coupling element 740, the channel 632 of the coupling member 630 may be configured to removably receive the coupling element 740 of the eye shield component 700 so that the eye shield component 700 is removably coupled to the nose/mouth component 600 of the second embodiment of the facemask 500. While only one coupling member and one coupling element is illustrated in FIG. 11C, because coupling elements 730, 740 are substantially similar to one another, the coupling element 740 illustrated in FIG. 11C and associated description applies to both coupling elements 730, 740 and how they are received by the coupling members 620, 630, respectively. As illustrated in FIG. 11C, the coupling element 740 may be elongated having a proximal end 742 and a distal end 744. The proximal end 742 of the coupling element 740 may be coupled to the bottom edge 708 of the eye shield component 700, while the distal end 744 of the coupling element 740 may be in the shape of an inverted T, which is similar in shape to that of the channels 622, 632 of the coupling members 620, 630 of the nose/mouth component 600. Thus, as illustrated in FIG. 11C, the distal end 744 of the coupling element 740 may be received by the channel 632 of the coupling member 630 (and the distal end 734 of the other coupling element 730 may be similarly received by the channel 622 of the coupling member 620) to removably couple the eye shield component 700 to the nose/mouth component 600.

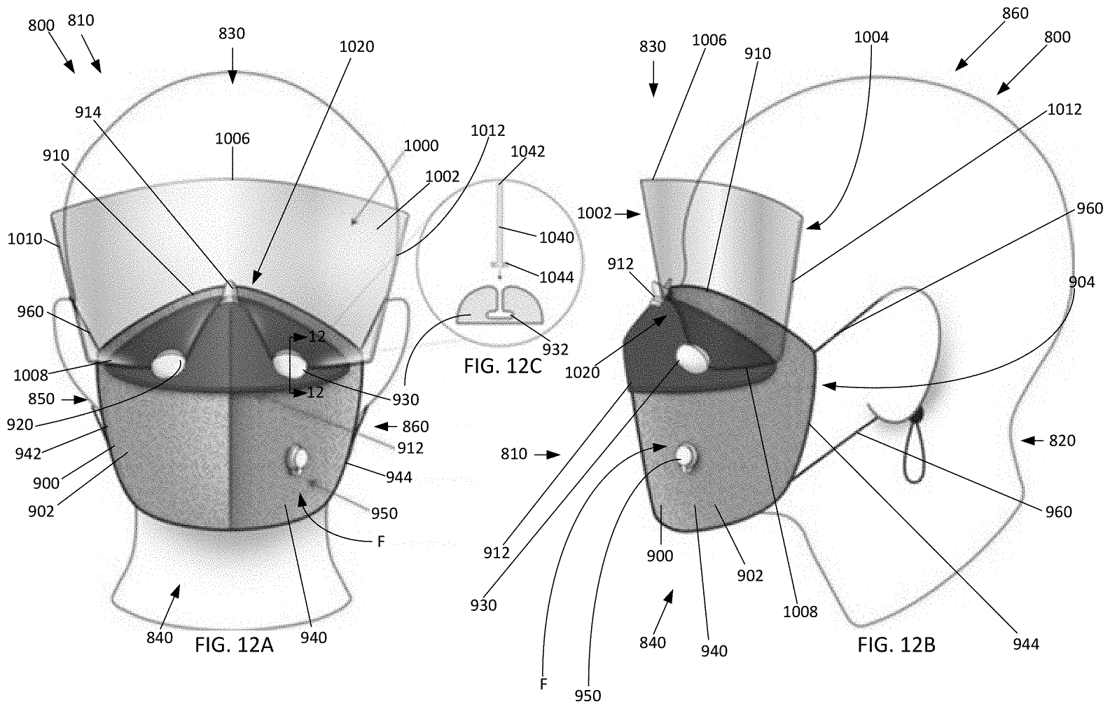

Turning to FIGS. 12A and 12B, illustrated is a third embodiment of a facemask 800 configured to be worn by a user. The third embodiment of the facemask 800 may contain similarities to that of the first and second embodiments of the facemask 100, 500, but may also contain some differences from the first and second embodiments of the facemask 100, 500. As illustrated in FIGS. 12A and 12B, the facemask 800 includes an front side or exterior side 810 and an opposite rear side or interior side 820. The interior side 820 may primarily face toward the user, while the exterior side 810 may primarily face away from the user. The third embodiment of the facemask 800 may further include a top side 830, an opposite bottom side 840, a first side 850, and an opposite second side 860. As further illustrated in FIGS. 12A and 12B, the facemask 800 may include a first component 900, also referred to herein as a lower component or nose/mouth component, and a second component 1000, also referred to herein as an upper component or eye shield component.

The nose/mouth component 900 of the third embodiment of the facemask 800 may be configured to cover the nose, mouth, and surrounding facial area of a user when the facemask 800 is worn by the user. As illustrated in FIGS. 12A and 12B, the nose/mouth component 900 includes an exterior side 902 and an opposite interior side 904. The exterior side 902 may primarily face away from the user, while the interior side 904 may primarily face toward the user when the facemask 800 is worn by the user. Unlike the first and second embodiments of the facemask 100, 500, the nose/mouth component 900 of the third embodiment of the facemask 800 may be formed or constructed from primarily a fabric material. Thus, the third embodiment of the facemask 800 may be a “fabric mask” that is configured to cover the nose and mouth area of a user's face. As illustrated in FIGS. 12A and 12B, an upper portion 910 of the nose/mouth component 900 of the facemask 800 may contain a flexible membrane 912. The flexible membrane 912 may be constructed from any flexible and deformable material including, but not limited to, rubber, synthetic rubber, silicone, polyvinyl chloride, polyurethane, etc. The flexible membrane 912 may be disposed on and affixed to the exterior side 902 of the nose/mouth component 900 in the upper portion 910 of the nose/mouth component 900 via any known affixing methods including, but not limited to, bonding, adhesives, stitching, hook and loop fasteners, etc.

Disposed on the flexible membrane 912 may be a bridge securement member 914 that functions in much of the same manner as the nose bridges 212, 612 and securement slots 214, 614 of the first and second embodiments of the facemasks 100, 500, respectively. That is, the bridge securement member 914 may contain a linear slot (not illustrated) that extends in a transverse direction to the facemask 800 (i.e., the slot of the bridge securement member 914 may extend across the bridge securement member 914 in a direction spanning generally from the first side 850 of the facemask 800 to the second side 860 of the facemask 800). The slot may be configured to receive a portion of the eye shield component 1000, as explained in further detail below, when the eye shield component 1000 is coupled to the nose/mouth component 900. The bridge securement member 914 may also be constructed from rubber or a rubber-like material such that the bridge securement member 914 has a degree of resiliency or deformability that may enable the bridge securement member 914 to form a friction fit with the portion of the eye shield component 1000 disposed within the slot of the bridge securement member 914. In other words, the material of the bridge securement member 914 may be configured to impart friction forces onto a portion of the eye shield component 1000 to prevent the eye shield component 1000 from shifting/moving with respect to the nose/mouth component 900.

With continued reference to FIGS. 12A and 12B, and with further reference to FIG. 12C, a first coupling member 920 and a second coupling member 930, similar to the coupling members 220, 230, 620, 630 of the first and second embodiments of the facemask 100, 500, may also be disposed on the flexible membrane 912 of the nose/mouth component 900. The first coupling member 920 may be disposed on the flexible membrane 912 more proximate to the first side 850 of the facemask 800 than the second coupling member 930, while the second coupling member 930 may be disposed more proximate to the second side 860 of the facemask 800 than the first coupling member 920. While FIG. 12C only illustrates coupling member 930, because the coupling members 920, 930 are substantially similar to one another, the illustrated cross-section and associated description applies to both coupling members 920, 930. As illustrated in FIG. 12C, the coupling member 930 may include an inverted T-shaped channel 932 (and coupling member 920 may include an identical inverted T-shaped channel 922). The channels 922, 932 may span or extend through the coupling members 920, 930, respectively, in a transverse direction to the facemask 800 (i.e., the channels 922, 932 extend through the coupling members 920, 930, respectively, in a direction spanning generally from the first side 850 of the facemask 800 to the second side 860 of the facemask 800). Like that explained previously for the channels 222, 232 of the coupling members 220, 230 of the first embodiment of the facemask 100 and the channels 622, 632 of the coupling members 620, 630 of the second embodiment of the facemask 500, the channels 922, 932 of the coupling members 920, 930 may be configured to receive portions of the eye shield component 1000 when the eye shield component 1000 is coupled to the nose/mouth component 900. The coupling members 920, 930 may be constructed from rubber or a similar material such that the coupling members 920, 930 have a degree of resiliency or deformability, while also being capable of forming a friction fit with the portions of the eye shield component 1000 disposed within the channels 922, 932 of the coupling members 920, 930, respectively. In other words, the material of the coupling members 920, 930 may impart friction forces onto portions of the eye shield component 1000 to prevent the eye shield component 1000 from shifting/moving with respect to the nose/mouth component 900.

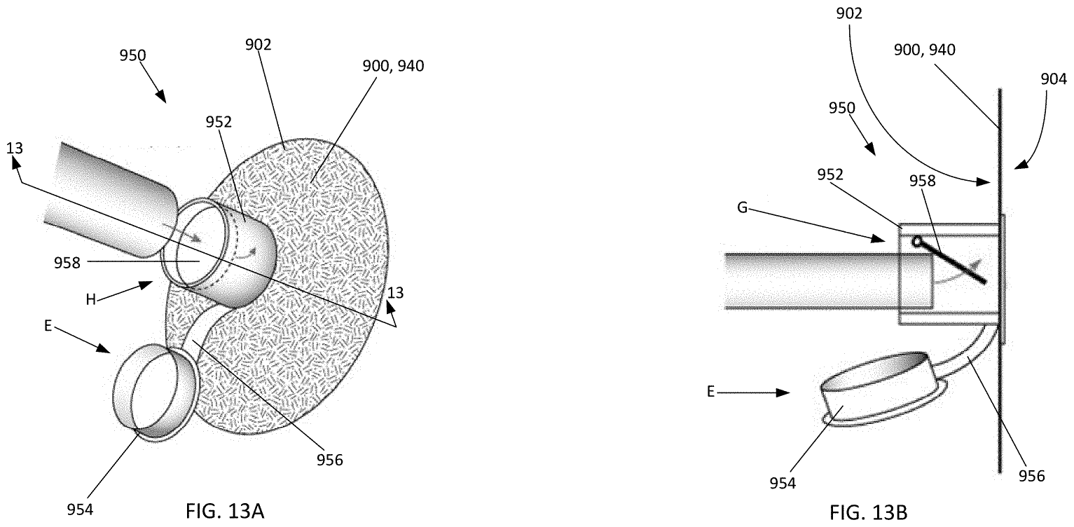

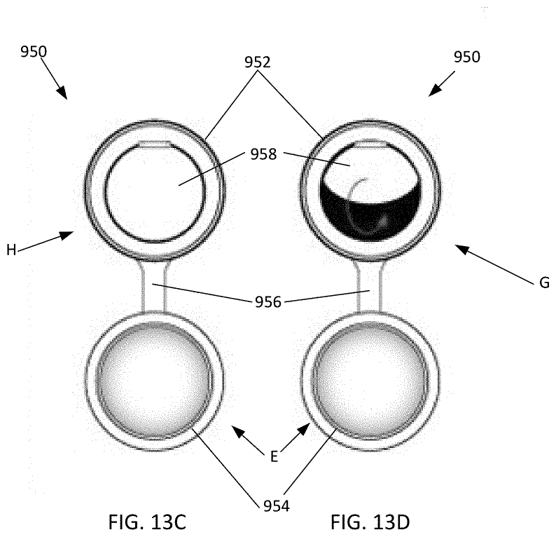

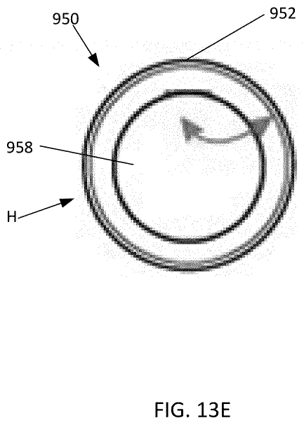

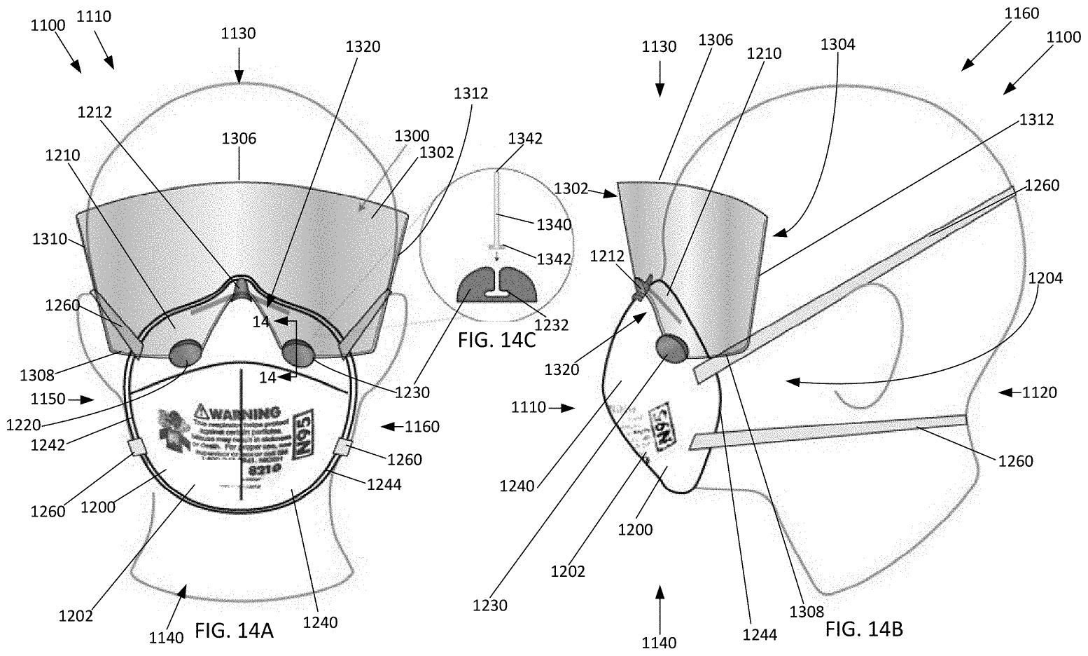

With continued reference to FIGS. 12A and 12B, the nose/mouth component 900 further includes a mouth portion 940, which includes a first side 942 and an opposite second side 944. As illustrated, the mouth portion 940 may further include a straw access 950. While the straw access 950 is illustrated as being disposed more proximate to the second side 944 of the mouth portion 940 than the first side 942 of the mouth portion 940, the straw access 950 may be located in any position/location on the mouth portion 940 of the nose/mouth component 900. The straw access 950 is further illustrated in FIGS. 13A-13E. The straw access 950 may include a conduit 952 that, as best illustrated in FIG. 13B, may span from the exterior side 902 of the nose/mouth component 900, through the nose/mouth component 900, to the interior side 904 of the nose/mouth component 900. The conduit 952 of the straw access 950 may be sized to receive a straw of a drink to enable a user of the facemask 800 to consume a beverage without having to remove the facemask 800 from their face or pull the facemask 800 away from their face. As illustrated, straw access 950 may further include a cover 954 that may be configured to cover/seal an end of the conduit 952 of the straw access 950. The cover 954 may be hingedly coupled to the mouth portion 940 or the conduit 952 via a tether, lanyard, or strap 956. In some other embodiments, the cover 954 may simply be removable from the conduit 952 of the straw access 950. The cover 954 may be repositioned between an open position E (shown in FIGS. 13A-13D), where the cover 954 does not cover the end of the conduit 952, and a closed position F (shown in FIGS. 12A and 12B), where the cover 954 is secured onto the end of the conduit 952 to form a seal for the conduit 952 of the straw access 950. The cover 954 may be formed from plastic, silicon, rubber, or any other similar impermeable material capable of forming a seal with the conduit 952.

As further illustrated in FIGS. 13A-13E, the straw access 950 may also include a hinged door 958 disposed within the conduit 952 of the straw access 950. The hinged door 958 may be configured to form an additional seal within the conduit 952 such that aerosols and droplets do not enter or exit the facemask 800 when a straw is not disposed within the conduit 952 of the straw access 950. As illustrated, the hinged door 958 may be repositionable between an opened position G (shown in FIGS. 13B and 13D) and a closed position H (shown in FIG. 13A, 13C, and 13E). The hinged door 958 may be a spring loaded or resilient door that is biased to the closed position H. As best illustrated in FIG. 13B, when a straw is inserted into the conduit 952 of the straw access 950, the end of the straw may reposition the hinged door 958 from the closed position H to the opened position E.

Turning back to FIGS. 12A and 12B, the third embodiment of the facemask 800 may also include one or more straps 960. The straps 960 may be coupled to the nose/mouth component 900 on the first and second sides 942, 944 of the mouth portion 940 of the facemask 800. Thus, as illustrated in FIGS. 12A and 12B, when the facemask 800 is worn by a user, each one of the straps 960 may be configured to wrap around one of the ears of the user to removably secure the facemask 800 to the user's face. In other embodiments, the straps 960 may be configured to wrap around the user's head to removably secure the facemask 800 to the user's face. The straps 960 may contain a degree of resiliency such that they are capable of stretching, and/or their length may be adjustable so that the fit of the facemask 800 on the user's face/head may be made comfortable.

Continuing with FIGS. 12A and 12B, and as previously explained, the third embodiment of the facemask 800 may include an eye shield component 1000 that is removably coupled to the nose/mouth component 900 of the facemask 800. The eye shield component 1000 may include an exterior surface 1002, which faces away from the user when the user is wearing the facemask 800, and an opposite interior surface 1004, which faces toward the user when the user is wearing the facemask 800. Like the eye shield components 300, 700 of the first and second embodiments of the facemask 100, 500, respectively, the eye shield component 1000 of the third embodiment of the facemask 800 may further include a top edge 1006, a bottom edge 1008 opposite the top edge 1006, a first side edge 1010, and a second side edge 1012 opposite the first side edge 1010. The eye shield component 1000 may have a transparency that enables a user to see objects and the surrounding environment through the eye shield component 1000. The eye shield component 1000 may be constructed from polycarbonate, plastic, or any other similar and suitable material.

Like the eye shield components 300, 700 of the first and second embodiments of the facemask 100, 500, respectively, the bottom edge 1008 of the eye shield component 1000 of the third embodiment of the facemask 800 may contain a central notch or cutout portion 1020 that is disposed along the bottom edge 1008 equidistant from the first and second side edges 1010, 1012 of the eye shield component 1000. The bottom edge 1008 of the eye shield component 1000 may further include a first coupling element 1030 and a second coupling element 1040 that descend from the bottom edge 1008 of the eye shield component 1000. As best illustrated in FIG. 12C, which is a cross-sectional view of the coupling member 930 and the coupling element 1040, the channel 932 of the coupling member 930 may be configured to removably receive the coupling element 1040 of the eye shield component 1000 so that the eye shield component 1000 is removably coupled to the nose/mouth component 900 of the third embodiment of the facemask 800. While only one coupling member and one coupling element is illustrated in FIG. 12C, because coupling elements 1030, 1040 are substantially similar to one another, the coupling element 1040 illustrated in FIG. 12C and associated description applies to both coupling elements 1030, 1040 and how they are received by the coupling members 920, 930, respectively. As illustrated in FIG. 12C, the coupling element 1040 may be elongated having a proximal end 1042 and a distal end 1044. The proximal end 1042 of the coupling element 1040 may be coupled to the bottom edge 1008 of the eye shield component 1000, while the distal end 1044 of the coupling element 1040 may be in the shape of an inverted T, which is similar in shape to that of the channels 922, 932 of the coupling members 920, 930 of the nose/mouth component 900. Thus, as illustrated in FIG. 12C, the distal end 1044 of the coupling element 1040 may be received by the channel 932 of the coupling member 930 (and the distal end 1034 of the other coupling element 1030 may be similarly received by the channel 922 of the coupling member 920) to removably couple the eye shield component 1000 to the nose/mouth component 900.

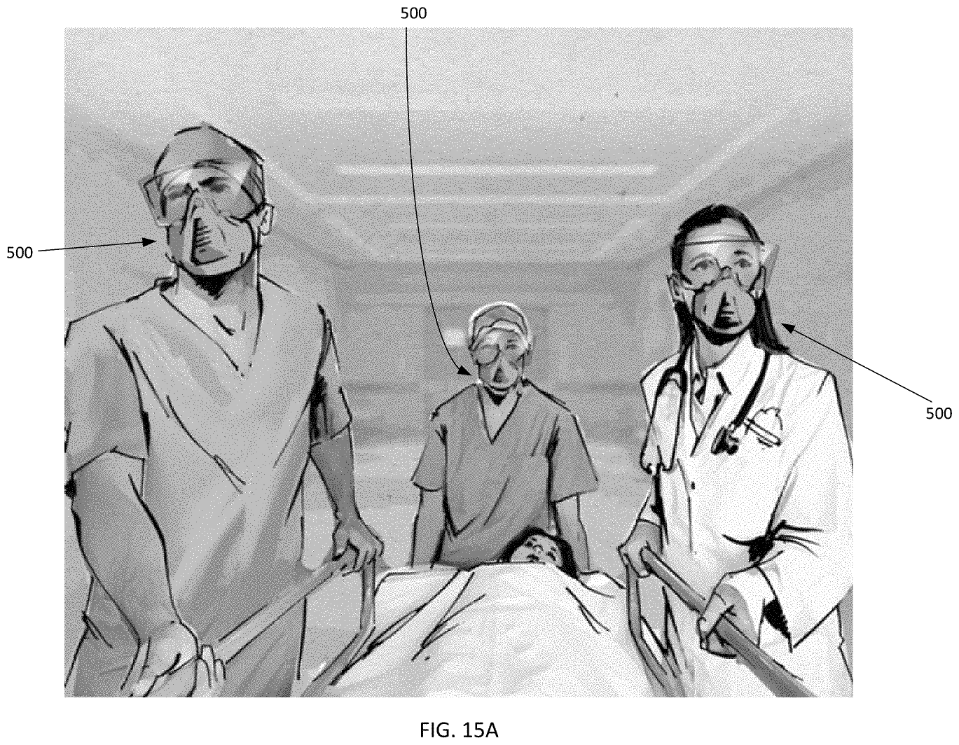

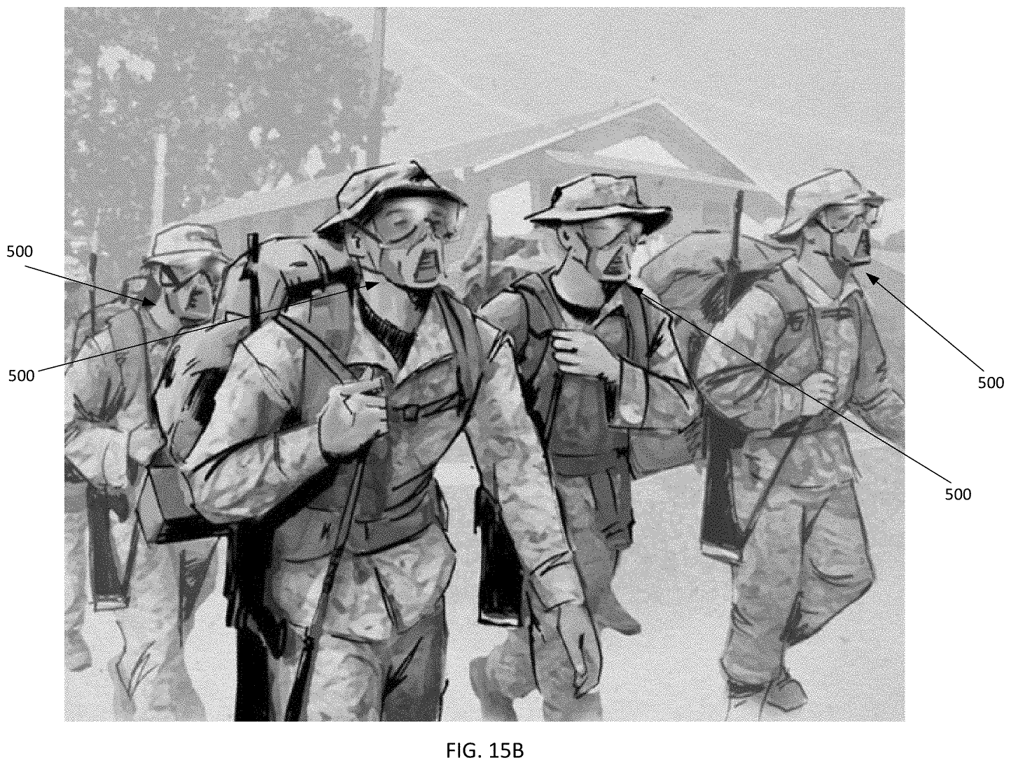

Turning to FIGS. 14A and 14B, illustrated is a fourth embodiment of a facemask 1100 configured to be worn by a user. The fourth embodiment of the facemask 1100 may contain similarities to that of the first, second, and third embodiments of the facemask 100, 500, 800 but may also contain some differences from the first, second, and third embodiments of the facemask 100, 500, 800. As illustrated in FIGS. 14A and 14B, the facemask 1100 includes an front side or exterior side 1110 and an opposite rear side or interior side 1120. The interior side 1120 may primarily face toward the user, while the exterior side 1110 may primarily face away from the user. The fourth embodiment of the facemask 1100 may further include a top side 1130, an opposite bottom side 1140, a first side 1150, and an opposite second side 1160. As further illustrated in FIGS. 14A and 14B, and like the previous embodiments, the facemask 1100 may include a first component 1200, also referred to herein as a lower component or nose/mouth component, and a second component 1300, also referred to herein as an upper component or eye shield component.

The nose/mouth component 1200 of the fourth embodiment of the facemask 1100 may be configured to cover the nose, mouth, and surrounding facial area of a user when the facemask 1100 is worn by the user. As illustrated in FIGS. 14A and 14B, the nose/mouth component 1200 includes an exterior side 1202 and an opposite interior side 1204. The exterior side 1202 may primarily face away from the user, while the interior side 1204 may primarily face toward the user when the facemask 1100 is worn by the user. Unlike the first, second, and third embodiments of the facemask 100, 500, 800, the nose/mouth component 1200 of the fourth embodiment of the facemask 1100 may be an N95 style mask (e.g., the nose/mouth component 1200 is constructed from a fine mesh of synthetic polymer fibers that form a nonwoven polypropylene fabric). As illustrated in FIGS. 14A and 14B, the nose/mouth component 1200 may contain an upper portion 1210 and a lower portion 1240. Disposed on the upper portion 1210 may be a nose bridge 1212 that contains a securement slot 1214 (not illustrated) that extends in a transverse direction to the facemask 1100 (i.e., the securement slot 1214 of the nose bridge 1212 may extend across the nose bridge 1212 in a direction spanning generally from the first side 1150 of the facemask 1100 to the second side 1160 of the facemask 1100). The nose bridge 1212 and the securement slot 1214 function in much of the same manner as the nose bridges 212, 612 and that securement slots 214, 614 of the first and second embodiments of the facemasks 100, 500, respectively. That is, the securement slot 1214 may be configured to receive a portion of the eye shield component 1300, as explained in further detail below, when the eye shield component 1300 is coupled to the nose/mouth component 1200. The nose bridge 1212 may also be constructed from rubber or a rubber-like material such that the nose bridge 1212 has a degree of resiliency or deformability that may enable the nose bridge 1212 to form a friction fit with the portion of the eye shield component 1300 disposed within the securement slot 1214 of the nose bridge 1212. In other words, the material of the nose bridge 1212 may be configured to impart friction forces onto a portion of the eye shield component 1300 to prevent the eye shield component 1300 from shifting/moving with respect to the nose/mouth component 1200.

With continued reference to FIGS. 14A and 14B, and with further reference to FIG. 14C, a first coupling member 1220 and a second coupling member 1230, similar to the coupling members 220, 230, 620, 630, 920, 930 of the first, second, and third embodiments of the facemask 100, 500, 800, may also be disposed on the upper portion 1210 of the nose/mouth component 1200. The first coupling member 1220 may be disposed on the upper portion 1210 more proximate to the first side 1150 of the facemask 1100 than the second coupling member 1230, while the second coupling member 1230 may be disposed more proximate to the second side 1160 of the facemask 1100 than the first coupling member 1220. While FIG. 14C only illustrates coupling member 1230, because the coupling members 1220, 1230 are substantially similar to one another, the illustrated cross-section and associated description applies to both coupling members 1220, 1230. As illustrated in FIG. 14C, the coupling member 1230 may include an inverted T-shaped channel 1232 (and coupling member 1220 may include an identical inverted T-shaped channel 1222). The channels 1222, 1232 may span or extend through the coupling members 1220, 1230, respectively, in a transverse direction to the facemask 1100 (i.e., the channels 1222, 1232 extend through the coupling members 1220, 1230, respectively, in a direction spanning generally from the first side 1150 of the facemask 1100 to the second side 1160 of the facemask 1100). Like that explained previously for the channels 222, 232 of the coupling members 220, 230 of the first embodiment of the facemask 100, the channels 622, 632 of the coupling members 620, 630 of the second embodiment of the facemask 500, and the channels 922, 932 of the coupling members 920, 930 of the third embodiment of the facemask 800, the channels 1222, 1232 of the coupling members 1220, 1230 may be configured to receive portions of the eye shield component 1300 when the eye shield component 1300 is coupled to the nose/mouth component 1200. The coupling members 1220, 1230 may be constructed from rubber or a similar material such that the coupling members 1220, 1230 have a degree of resiliency or deformability, while also being capable of forming a friction fit with the portions of the eye shield component 1300 disposed within the channels 1222, 1232 of the coupling members 1220, 1230, respectively. In other words, the material of the coupling members 1220, 1230 may impart friction forces onto portions of the eye shield component 1300 to prevent the eye shield component 1300 from shifting/moving with respect to the nose/mouth component 1200.

With continued reference to FIGS. 14A and 14B, and as previously explained, the nose/mouth component 1200 further includes a lower portion 1240, which includes a first side 1242 and an opposite second side 1244. The fourth embodiment of the facemask 1100 may also include one or more straps 1260. The straps 1260 may be coupled to the nose/mouth component 1200 on the first and second sides 1242, 1244 of the lower portion 1240 of the facemask 1100. Thus, as illustrated in FIGS. 14A and 14B, when the facemask 1100 is worn by a user, each one of the straps 1260 may be configured to wrap around the user's head to removably secure the facemask 1100 to the user's face. The straps 1260 may contain a degree of resiliency such that they are capable of stretching, and/or their length may be adjustable so that the fit of the facemask 1100 on the user's face/head may be made comfortable.

Continuing with FIGS. 14A and 14B, and as previously explained, the fourth embodiment of the facemask 1100 may include an eye shield component 1300 that is removably coupled to the nose/mouth component 1200 of the facemask 1100. The eye shield component 1300 may include an exterior surface 1302, which faces away from the user when the user is wearing the facemask 1100, and an opposite interior surface 1304, which faces toward the user when the user is wearing the facemask 1100. Like the eye shield components 300, 700, 1000 of the first, second, and third embodiments of the facemask 100, 500, 800, respectively, the eye shield component 1300 of the fourth embodiment of the facemask 1100 may further include a top edge 1306, a bottom edge 1308 opposite the top edge 1306, a first side edge 1310, and a second side edge 1312 opposite the first side edge 1310. The eye shield component 1300 may have a transparency that enables a user to see objects and the surrounding environment through the eye shield component 1300. The eye shield component 1300 may be constructed from polycarbonate, plastic, or any other similar and suitable material.

Like the eye shield components 300, 700, 1000 of the first, second, and third embodiments of the facemask 100, 500, 800, respectively, the bottom edge 1308 of the eye shield component 1300 of the third embodiment of the facemask 1100 may contain a central notch or cutout portion 1320 that is disposed along the bottom edge 1308 equidistant from the first and second side edges 1310, 1312 of the eye shield component 1300. The bottom edge 1308 of the eye shield component 1300 may further include a first coupling element 1330 and a second coupling element 1340 that descend from the bottom edge 1308 of the eye shield component 1300. As best illustrated in FIG. 14C, which is a cross-sectional view of the coupling member 1230 and the coupling element 1340, the channel 1232 of the coupling member 1230 may be configured to removably receive the coupling element 1340 of the eye shield component 1300 so that the eye shield component 1300 is removably coupled to the nose/mouth component 1200 of the fourth embodiment of the facemask 1100. While only one coupling member and one coupling element is illustrated in FIG. 14C, because coupling elements 1330, 1340 are substantially similar to one another, the coupling element 1340 illustrated in FIG. 14C and associated description applies to both coupling elements 1330, 1340 and how they are received by the coupling members 1220, 1230. As illustrated in FIG. 14C, the coupling element 1340 may be elongated having a proximal end 1342 and a distal end 1344. The proximal end 1342 of the coupling element 1340 may be coupled to the bottom edge 1308 of the eye shield component 1200, while the distal end 1344 of the coupling element 1340 may be in the shape of an inverted T, which is similar in shape to that of the channels 1222, 1232 of the coupling members 1220, 1230 of the nose/mouth component 1200. Thus, as illustrated in FIG. 14C, the distal end 1344 of the coupling element 1340 may be received by the channel 1232 of the coupling member 1230 (and the distal end 1334 of the other coupling element 1330 may be similarly received by the channel 1222 of the coupling member 1220) to removably couple the eye shield component 1300 to the nose/mouth component 1200.

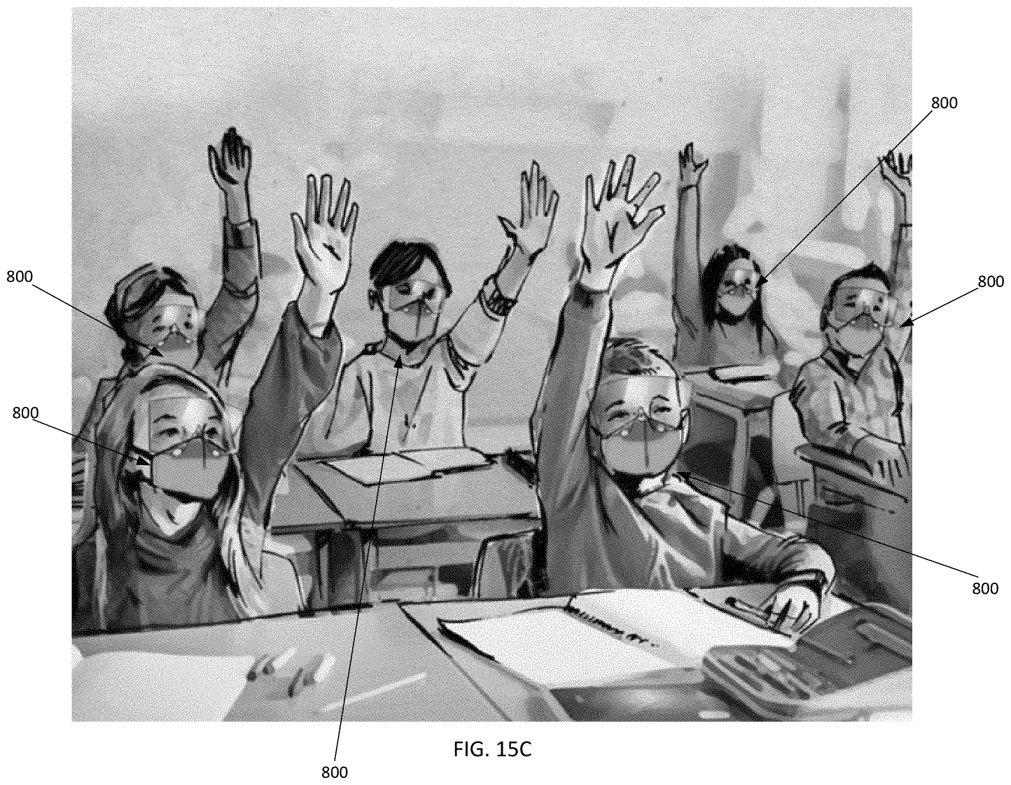

Turning to FIGS. 15A-15C, illustrated are various field uses of the second and third embodiments of the facemask 500, 800. For example, FIG. 15A illustrates the second embodiment of the facemask 500 being worn by healthcare workers in a hospital setting. FIG. 15B illustrates the second embodiment of the facemask 500 being worn by military personnel. Meanwhile, FIG. 15C illustrates the third embodiment of the facemask 800 being worn by children in a classroom school setting.

The facemasks 100, 500, 800, 1100 presented herein may be worn by a user to provide protection against aerosols and droplets in the air around the user, and dispensed into the air by other people present in the environment of the user. The shape and materials of the nose/mouth components 200, 600, 900, 1200 of the facemasks 100, 500, 800, 1100 may cause the nose/mouth components 200, 600, 900, 1200 to form a protective seal around the nose and mouth of the user of the facemasks 100, 500, 800, 1100. For the first embodiment of the facemask 100, the nose/mouth component 200 forms a protective seal around the nose and mouth such that the air breathed in by the user of the facemask 100 is filtered by the filter 270 located within the filter enclosure 260 in order to prevent the user of the facemask from inhaling or ingesting hazardous aerosols and/or droplets. In addition, the seal/covering formed by the nose/mouth components 200, 600, 900, 1200 of the facemasks 100, 500, 800, 1100 further ensure that any aerosols and droplets exhaled by the user of the facemasks 100, 500, 800, 1100 to do not escape the facemasks 100, 500, 800, 1100. In some instances, like that of the first embodiment of the facemask 100, the air exhaled by the user of the facemask 100 is filtered through the filter 270 of the filter enclosure 260 to prevent the user from exhaling hazardous aerosols and/or droplets in to the air surrounding the user. The baffles 266 of the filter enclosure 260 of the first embodiment of the facemask 100 further ensure that any air/gas passing through the filter enclosure 260, whether or not that air/gas contains any aerosols and/or droplets, is directed downward toward the ground. As previously explained, the baffles 266 further ensure that exhaled air does not rise upward toward the eye shield component 300, thus, reducing any fogging of the eye shield component 300 from user exhaled air/gas.

The first and third embodiments of the facemask 100, 800 presented herein may also enable a user of the facemasks 100, 800 to consume food and beverages without having to remove the facemasks 100, 800 or pull the facemask down/away from their face. As previously explained, the straw access 246, 950 of the facemasks 100, 800 may enable a straw to be inserted through the facemasks 100, 800 so that a user may sip a beverage via the straw inserted through the straw access 246, 950 while wearing the facemasks 100, 800. In addition, as previously explained with regard to the first embodiment of the facemask 100, the filter enclosure 260 may be hingedly coupled to the nose/mouth component 200 of the facemask 100, or otherwise removably or repositionably coupled to the nose/mouth component 200 of the facemask 100, such that the filter enclosure 260 may be either disposed within the central opening 250 of the facemask 100 (i.e., the closed position A) or disposed out of the central opening 250 of the facemask 100 (i.e., the open position B). When the filter enclosure 260 is repositioned to the open position B, where it is no longer disposed within the central opening 250, the user of the facemask 100 may consume food or beverages via the central opening 250 without removing the facemask 100 from their head. Once they have finished consuming the food and/or beverage, the user may quickly and easily reposition the filter enclosure 260 to the closed position A, where the filter enclosure 260 is disposed within the central opening 250.

As further explained above, the embodiments of the facemasks 100, 500, 800, 1100 presented herein also provides protection to the eyes of the user of the facemasks 100, 500, 800, 1100 through their corresponding eye shield components 300, 700, 1000, 1300. The eye shield components 300, 700, 1000, 1300 are configured to protect the eyes of the user from objects, droplets, and/or aerosols directed toward the user (i.e., coughing, sneezing, etc. by others in proximity to the user). The eye shield components 300, 700, 1000, 1300 may be easily detached from the nose/mouth components 200, 600, 900, 1200, respectively, when the user has a desire to wear the facemask 100, 500, 800, 1100 without the eye shield components 300, 700, 1000, 1300, when the user desires to interchange the eye shield components 300, 700, 1000, 1300 for other eye shield components 300, 700, 1000, 1300, or simply when the user wishes to wash the eye shield components 300, 700, 1000, 1300. As further explained, the eye shield components 300, 700, 1000, 1300 may couple to the nose/mouth components 200, 600, 900, 1200, respectively such that the facemasks 100, 500, 800, 1100 may be worn by users that wear glasses without the eye shield components 300, 700, 1000, 1300 interfering with the glasses of the user.

It is to be understood that terms such as “left,” “right,” “top,” “bottom,” “front,” “rear,” “side,” “height,” “length,” “width,” “upper,” “lower,” “interior,” “exterior,” “inner,” “outer” and the like as may be used herein, merely describe points or portions of reference and do not limit the present invention to any particular orientation or configuration. Further, the term “exemplary” is used herein to describe an example or illustration. Any embodiment described herein as exemplary is not to be construed as a preferred or advantageous embodiment, but rather as one example or illustration of a possible embodiment of the invention.

Although the disclosed inventions are illustrated and described herein as embodied in one or more specific examples, it is nevertheless not intended to be limited to the details shown, since various modifications and structural changes may be made therein without departing from the scope of the inventions and within the scope and range of equivalents of the claims. In addition, various features from one of the embodiments may be incorporated into another of the embodiments. Accordingly, it is appropriate that the appended claims be construed broadly and in a manner consistent with the scope of the disclosure as set forth in the following claims.

What is claimed is:

1. A facemask configured to be worn by a user, the facemask comprising:
a first portion configured to cover a nose and a mouth of the user when the facemask is worn by the user; and
a second portion removably coupled to the first portion, the second portion being positioned proximate to eyes of the user when the second portion is coupled to the first portion and the facemask is worn by the user.

2. The facemask of claim 1, wherein the second portion is a transparent shield that further comprises:
at least one coupling element descending from a bottom edge of the second portion.

3. The facemask of claim 2, wherein the first portion further comprises:
at least one coupling member configured to receive the at least one coupling element of the second portion when the second portion is removably coupled to the first portion.

4. The facemask of claim 3, wherein the at least one coupling member of the first portion is constructed from a material that both promotes a degree of deformability of the at least one coupling member and is configured to impart friction forces on the at least one coupling element received by the at least one coupling member when the second portion is removably coupled to the first portion.

5. The facemask of claim 3, wherein the at least one coupling element is a first coupling element descending from the bottom edge of the second portion proximate to a first side edge of the second portion, the second portion further comprising:
a second coupling element descending from the bottom edge of the second portion proximate to a second side edge of the second portion, the second side edge being opposite the first side edge.

6. The facemask of claim 5, wherein the at least one coupling member is a first coupling member disposed on the first portion more proximate to a first side of the first portion than a second side of the first portion, the first portion further comprising:
a second coupling member disposed on the first portion more proximate to the second side of the first portion than the first side of the first portion, the first coupling member being configured to receive the first coupling element and the second coupling member being configured to receive the second coupling element when the second portion is removably coupled to the first portion.

7. The facemask of claim 6, wherein the bottom edge of the second portion further comprises:
a central notch disposed between the first coupling element and the second coupling element, the central notch being disposed equidistant from the first side edge and the second side edge.

8. The facemask of claim 7, wherein the first portion further comprises:
a securement slot disposed between the first coupling member and the second coupling member, the central notch of the second portion being disposed within the securement slot when the second portion is removably coupled to the first portion.

9. A facemask configured to be worn by a user, the facemask comprising:
a lower component including:
a mouth portion that is configured to cover a mouth of the user when the facemask is worn by the user, and
a nose portion disposed above the mouth portion, the nose portion being configured to cover a nose of the user when the facemask is worn by the user; and
an upper component removably coupled to the nose portion of the lower component, the upper component being positioned proximate to eyes of the user when the upper component is coupled to the lower component and the facemask is worn by the user.

10. The facemask of claim 9, wherein the upper component is a transparent shield that further comprises:
at least one coupling element descending from a bottom edge of the upper component.

11. The facemask of claim 10, wherein the lower component further comprises:
at least one coupling member disposed on the nose portion, the at least one coupling member being configured to receive the at least one coupling element of the upper component when the upper component is removably coupled to the lower component.

12. The facemask of claim 11, wherein the at least one coupling member of the lower component is constructed from a material that both promotes a degree of deformability of the at least one coupling member and is configured to impart friction forces on the at least one coupling element received by the at least one coupling member when the upper component is removably coupled to the lower component.

13. The facemask of claim 9, wherein the lower component further comprises:
a central opening disposed in the mouth portion of the lower component such that the central opening is disposed proximate to the mouth of the user when the facemask is worn by the user; and
a filter enclosure rotatably coupled to mouth portion of the lower component proximate to the central opening, the filter enclosure being rotatable between a closed position, where the filter enclosure is disposed within the central opening, and an open position, where the filter enclosure is rotated away from the central opening.

14. The facemask of claim 9, wherein the lower component further comprises:
a straw access opening disposed in the mouth portion of the lower component, the straw access opening being configured to receive a straw; and
a cover coupled to the straw access opening, the cover being reconfigurable between an uncovered position, where the user may insert the straw into the straw access opening, and a covered position, where the cover is disposed over the straw access opening.

15. A facemask configured to be worn by a user, the facemask comprising:
a nose and mouth component configured to cover a nose and a mouth of the user when the facemask is worn by the user; and
an eye shield component removably coupled to the nose and mouth component, the eye shield component being positioned proximate to eyes of the user when the eye shield component is coupled to the nose and mouth component and the facemask is worn by the user.

16. The facemask of claim 15, wherein:
the eye shield component is transparent and further comprises at least one coupling element descending from a bottom edge of the eye shield component; and
the nose and mouth component further comprises at least one coupling member configured to receive the at least one coupling element of the eye shield component when the eye shield component is removably coupled to the nose and mouth component.

17. The facemask of claim 16, wherein the at least one coupling member of the nose and mouth component is constructed from a material that both promotes a degree of deformability of the at least one coupling member and is configured to impart friction forces on the at least one coupling element received by the at least one coupling member when the eye shield component is removably coupled to the nose and mouth component.

18. The facemask of claim 17, wherein the nose and mouth component is constructed from an impermeable and waterproof material.

19. The facemask of claim 17, wherein the nose and mouth component is constructed from a fabric and a flexible membrane disposed on an exterior surface of the fabric.

20. The facemask of claim 17, wherein the nose and mouth component is constructed from a mesh of synthetic polymer fibers.
